SEQUENCE LISTING (35 sequences)

SEQ ID NO: 1 - DNA, 4506 bp, Saccharomyces cerevisiae
  source 1..4506: mol_type = genomic DNA
atgagcaatatcaaaagcacgcaagatagctctcataatgctgtcgctagaagctcaagcgcttcttttgcagcttcagaagaatcatttacgggcataacccatgacaaagatgagcagagcgataccccggcggataaactaacaaaaatgctgacaggacctgcaagagacactgcgagccagattagtgccactgtgtctgaaatggcgccagatgtcgtatctaaagtggagtcatttgcagatgcactatcccgtcatacaacgagaagcggtgcctttaatatggattcagatagtgacgatgggttcgatgcccatgccatctttgaaagttttgtaagagacgctgatgagcaaggcatccatatccgcaaggctggtgttaccatagaggacgtaagcgctaaaggtgtggatgcgagtgccctagaaggtgctacctttggtaacattctttgtttaccgttgaccatctttaaaggtattaaggctaagaggcatcaaaagatgagacagatcataagcaatgtcaatgccctggcagaagcgggtgaaatgattttggttcttggaaggcctggtgctggttgttcctcctttttaaaagtaacagctggtgaaatagatcagtttgccggtggtgtttccggtgaagtagcatatgatggtattccccaagaagaaatgatgaaacgatataaagcagatgttatttacaatggtgagttggatgttcatttcccttatttaacagttaagcaaactttggatttcgctattgcctgcaaaacgcctgctctcagagtcaataacgtttccaaaaaggaatacattgcatccagaagagatttatatgcaaccattttcggtctaaggcatacctataataccaaagttggtaacgatttcgttagaggtgtatctggtggtgaacgtaagcgtgtttccattgccgaggctttggcagccaaaggttccatttactgttgggataatgccactagaggtttggatgcgtctacggccttagaatacgcaaaagccatccgtattatgacaaacttattgaaatcaaccgcttttgttacaatttatcaggcaagtgaaaacatttacgaaacatttgataaagtcactgtcctttattctggtaagcaaatttattttggtttgatccacgaggcaaaaccttatttcgcaaaaatgggttatttgtgtcctccaaggcaagcaacagctgaatttttaaccgcgttgactgatccaaatggattccatctgatcaagccaggttatgaaaataaagtaccaagaaccgctgaggaattcgaaacatattggttaaattctccagagtttgctcaaatgaaaaaagatatcgctgcttataaagagaaggtcaataccgaaaagactaaagaagtttatgacgaatcgatggctcaagagaaatccaaatatacgagaaagaagtcttattatacagtgtcatattgggaacaagttaaactgtgtacccaacgtgggttccaaagaatttacggtaacaagagttatacagtcatcaatgtctgctctgcaataattcaatcttttattactggatcattattttacaataccccttcatccacttccggtgctttttcaagaggtggtgtgttgtattttgcgctactatattattctttgatgggactggcgaatatttcttttgaacataggccaatcttacaaaagcacaagggctattctttgtatcatccttcagctgaggcaattggctccactctggcatctttccccttcagaatgattggtttgacctgtttctttatcattttattcttcctatctgggttgcacagaacagcgggatcattttttaccatctatttgttcttaaccatgtgttcagaggcgatcaatggtttatttgagatggtttcttcagtatgtgacactctttctcaagctaactctatctcgggtattctgatgatgtctatctcaatgtactctacctatatgatccaattgccttcgatgcatccatggtttaaatggatatcgtacgtactacctatcaggtacgccttcgagtcgatgttaaatgccgaatttcacggtaggcatatggattgtgctaacactctagtacccagtggaggagactatgataatttatccgatgactacaaagtatgtgcttttgttggttcgaaaccaggtcagtcttatgtgcttggtgatgactaccttaaaaatcaatttcagtacgtttataagcacacgtggagaaactttggtatcttgtggtgctttttactgggttatgttgttttgaaagtgatattcacagaatataagaggcctgtgaaaggtggtggtgatgctcttatcttcaagaaaggatcaaaaagatttatcgcacatgcagatgaagaatctccagacaatgtcaatgatatagatgccaaagagcaattctccagtgaaagtagcggcgcaaatgatgaagtatttgatgatttagaagccaaaggtgttttcatttggaaggacgtatgctttactattccatatgaaggcggtaagagaatgcttttggataatgtttcaggttattgtattccaggtaccatgacggccttgatgggagagtcaggtgctggtaaaacaactttgttaaatactcttgctcaaagaaatgtcggtatcattactggtgatatgcttgtcaatggacgtcccattgatgcgagtttcgaaaggcgtacaggttatgtacaacaacaggatatacatatcgcagagttaactgttagggaatcgttgcagttttctgctcgtatgcgtcgccctcagcatttgcctgattctgaaaaaatggattatgtggaaaaaatcatcagagttttgggaatggaagagtatgcggaagcccttgttggtgaggttggttgtggtttaaacgttgaacagagaaagaagctgtctattggtgttgaactagtcgccaaaccagacttattattattcctcgatgaacctacatcaggtttggattctcaatcttcatgggccattattcaattattaagaaagttatcaaaagctggccaatccattctttgtacgatccatcaaccttcagctactctgttcgaagagtttgatagattactacttttgaggaagggtggacaaactgtttatttcggagatattggtaagaactctgccaccattttgaactactttgaaaggaatggggcaagaaaatgtgattctagtgaaaatcctgctgaatatattttagaggctattggtgccggtgccacagcatccgtcaaagaagactggcacgaaaaatggttgaactctgtcgagtttgaacaaacaaaagaaaaagtacaggatttaataaatgatttatcgaaacaagaaactaaatccgaagttggagacaaaccttccaaatatgctacttcttatgcttaccagttcagatatgttttaatcagaacctctacttcattttggagaagtctgaattacatcatgtcaaagatgatgctaatgctggttggtggtctgtatattggtttcacatttttcaatgttggtaaaagttatgtcggcttacaaaatgcgatgttcgcggcatttatctctattatcttgtctgctcctgcaatgaaccaaatccaaggacgtgctattgcctccagagaactttttgaagttagggaatcccaatctaacatgtttcactggtcgctggtgttgatcactcagtacttgagcgaacttccctatcatttatttttttcgacaattttctttgtctcatcgtattttccattaagaatcttcttcgaagcgtcaagatctgcggtgtactttttgaattactgcattatgttccagttatactatgttggtcttggcttaatgatcctatatatgtcaccgaaccttccatccgctaatgttatcttaggtttgtgtctgtcatttatgctttctttctgtggtgttacacaacctgtctcattgatgcctggcttctggacattcatgtggaaggcttccccatacacatattttgttcagaatctggtcggaattatgctgcacaaaaaaccagtcgtatgcaaaaagaaagaactaaactacttcaacccaccaaacggctcaacgtgtggagagtacatgaaaccctttttggaaaaagctactggttacatcgaaaatcctgatgctacgtcagattgtgcatactgtatttacgaagttggagataattatttgacacatatcagctctaagtatagctacttgtggagaaattttggaatattttggatttacattttcttcaatatcattgctatggtttgtgtgtattacctcttccatgtaagacaatcttccttcctaagccccgtatctatactcaataaaattaaaaacataaggaaaaagaagcagtaa

SEQ ID NO: 2 - DNA, 4536 bp, Saccharomyces cerevisiae
  source 1..4536: mol_type = other DNA
atgtcttcgactgacgaacatattgagaaagacatttcgtcgagatcgaaccatgacgatgattatgctaattcggtacaatcctacgctgcctccgaaggccaagttgataatgaggatttggcagccacttctcagctatcccgtcacctttcaaacattctttccaatgaagaaggtattgaaaggttggagtctatggcgagagtcatttcacataagacaaagaaggaaatggactcttttgaaattaatgacttagattttgatttgcgctcactattacattatttgaggtctcgtcaattggaacagggaattgaacctggtgattctggtattgcctttaaaaacctaacagcagtcggtgttgatgcctctgctgcatatgggcctagtgttgaagagatgtttagaaatattgctagtataccggcacatctcataagtaaatttaccaagaaatctgatgtcccattaaggaatattattcaaaattgtacgggtgtcgttgaatctggtgaaatgttatttgtcgtcggtaggccaggtgcaggttgctccactttcctaaagtgtctatctggtgaaacttcagaattagttgatgtacaaggtgaattctcctatgatggtctggaccaaagcgaaatgatgtctaagtataaaggttacgttatttactgtcccgagcttgatttccatttcccaaaaattactgtgaaggaaacaatcgattttgccctaaaatgtaagactcctcgtgttagaattgacaaaatgacgagaaagcaatacgttgataacatcagagatatgtggtgtaccgtttttggtttaagacacacatatgccaccaaagtcggtaacgatttcgtaagaggtgtttctggtggtgaacgtaagcgtgtttccttggttgaagctcaggcaatgaatgcctccatctactcttgggataacgccacaagaggtttggatgcctctactgctttagagtttgcccaagccattagaacggctacaaatatggtaaacaactctgctattgttgctatttaccaagctggtgaaaatatttatgaattatttgataaaactactgttctatataacggtagacagatttacttcggtcctgctgataaagctgttggatatttccaaagaatgggttgggttaaaccaaacagaatgacctctgcggaatttttaacatccgtcacggtcgattttgaaaataggacattggatattaaacctggctatgaagataaagttccaaaatctagttcagagtttgaggaatactggttgaactctgaggattatcaggaacttttaagaacttatgatgattatcaaagtagacaccctgttaatgaaacgagagatagactggatgtggccaagaagcaaagactgcaacaaggccaaagagaaaattctcaatatgttgtcaattattggacacaagtttattattgtatgattcgtggttttcaaagggttaagggtgattcaacgtatactaaggtctacttaagttcttttttgatcaaagctttgattatcggttctatgttccacaaaattgatgacaaaagtcaatccaccacggcaggtgcttattctcgtggtggtatgttattctatgttttattgttcgcttctgttacttccttggccgaaattggtaactctttttctagtagacctgttattgtcaaacacaaatcatattccatgtaccatttgtctgcggaatcgttacaagagattatcactgagttccctactaaatttgtcgctattgtgatactatgtttgattacttactggattccatttatgaaatatgaagctggtgctttcttccagtatattttatatctactgactgtgcaacaatgtacttctttcattttcaagtttgttgctactatgagtaaatctggtgtggatgcccatgccgtcggtggtttatgggtcctgatgctttgtgtttatgctggttttgtcttgccaattggtgaaatgcatcattggattagatggcttcatttcattaatcctttaacttatgcttttgaaagtttagtttccactgaatttcaccacagggaaatgttgtgtagcgccttagtcccatctggtcctggttatgaaggtatttctattgctaaccaagtctgtgatgctgctggtgcggttaagggtaacttgtatgttagcggtgactcttacatcttacaccaatatcatttcgcatataagcatgcttggagaaattggggtgtgaacattgtgtggacttttggttatattgtgttcaatgtcatcttatcagaatatttgaaacctgttgagggaggaggtgacttgctgttatataagagaggtcatatgccggagttaggtaccgaaaatgcagatgcaagaaccgcttccagagaggaaatgatggaggctctgaatggtccaaatgtcgatttagaaaaggtcattgcagaaaaggacgttttcacctggaaccatctggactacaccattccatacgacggagctacaagaaaattattatcggatgtctttggttacgttaagcctggtaagatgaccgccttgatgggtgaatccggtgctggtaaaactaccttgttaaatgttttagcacaaagaatcaatatgggtgtcatcactggtgatatgttagtcaatgccaagcccttgcctgcttctttcaacagatcatgtggttatgttgcgcaagccgataatcatatggccgaattatctgttagggaatccctgagatttgcagccgagttaagacagcaaagttccgttccgttagaggagaaatatgaatatgttgaaaaaattatcacattgctaggtatgcaaaattacgctgaagccttagttggtaagactggtagaggtttgaacgttgaacagagaaagaagttatctattggtgttgaactggttgctaaaccatcattattattgtttttggatgagcctacctctggtctggactctcagtctgcttggtcaattgttcaattcatgagagccttagctgattctggtcaatccattttgtgtacgattcatcaaccctctgctaccttgtttgaacagtttgacagattgttgttgttaaagaaaggtggtaagatggtttactttggtgacattggtccaaattctgaaactttgttgaagtattttgaacgtcaatctggtatgaagtgtggtgtttctgaaaatccagctgaatatattttgaattgtattggtgccggtgccactgctagtgttaactctgattggcacgacttatggcttgcttccccagaatgtgccgctgcaagggctgaagttgaagaattacatcgtactttacctggtagagcagttaatgatgatcctgagttagctacaagatttgctgccagttacatgactcaaatcaaatgtgttttacgtagaacagctcttcaattttggagatcgcctgtctatatcagggccaaattctttgaatgtgtcgcatgtgctttgttcgtcggtttatcatatgttggtgtaaatcactctgttggtggtgccattgaggccttttcgtctattttcatgctattattgattgctctggctatgatcaatcaactgcacgtcttcgcttatgatagtagggaattatatgaggttagagaagccgcttctaacactttccattggagtgtcttgttattatgtcatgctgctgttgaaaacttttggtccacactttgtcagtttatgtgtttcatttgctactactggccagctcaattcagtggacgtgcatctcatgcaggtttcttcttcttcttctatgttttaattttcccattatattttgtcacatatggtctatggatcctgtacatgtctcctgatgttccctcagcttctatgattaattccaatttgtttgctgctatgttactgttctgtggtattttacaaccaagagagaaaatgcctgccttctggagaagattgatgtataatgtatcaccatttacctacgtggttcaagctttggttacaccattagttcacaataaaaaggtcgtttgtaatcctcatgaatacaacatcatggacccaccaagcggaaaaacttgtggtgagtttttatctacctatatggacaataataccggttatttggtaaatccaactgccaccgaaaactgtcaatattgcccatacactgttcaagatcaagttgtggctaaatacaatgtcaaatgggatcacagatggagaaactttggtttcatgtgggcttatatttgcttcaatattgccgctatgttgatttgttactatgttgtaagagttaaggtgtggtctttgaagtctgttttgaatttcaagaaatggtttaatgggccaagaaaggaaagacatgaaaaagataccaacattttccaaacagttccaggtgacgaaaataaaatcacgaagaaataa

SEQ ID NO: 3 - DNA, 4434 bp, Saccharomyces cerevisiae
  source 1..4434: mol_type = other DNA
atgacgattaccgtgggggatgcagtttcggagacggagctggaaaacaaaagtcaaaacgtggtactatctcccaaggcatctgcttcttcagacataagcacagatgttgataaggacacatcgtcttcttgggatgacaaatctttgctgcctacaggtgaatatattgtggacagaaataagccccaaacctacttgaatagcgatgatatcgaaaaagtgacagaatctgatattttccctcagaaacgtctgttttcattcttgcactctaagaaaattccagaagtaccacaaaccgatgacgagaggaagatatatcctctgttccatacaaatattatctctaacatgtttttttggtgggttctacccatcctgcgagttggttataagagaacgatacagccgaacgatctcttcaaaatggatccgaggatgtctatagagaccctttatgacgactttgaaaaaaacatgatttactattttgagaagacgaggaaaaaataccgtaaaagacatccagaagcgacagaagaagaggttatggaaaatgccaaactacctaaacatacagttctgagagctttattattcacttttaagaaacagtacttcatgtcgatagtgtttgcaattctcgctaattgtacatccggttttaaccccatgattaccaagaggctaattgagtttgtcgaagaaaaggctatttttcatagcatgcatgttaacaaaggtattggttacgctattggtgcatgtttgatgatgttcgttaacgggttgacgttcaaccatttctttcatacatcccaactgactggtgtgcaagctaagtctattcttactaaagctgccatgaagaaaatgtttaatgcatctaattatgcgagacattgttttcctaacggtaaagtgacttcttttgtaacaacagatctcgctagaattgaatttgccttatcttttcagccgtttttggctgggttccctgcaattttggctatttgcattgttttattgatcgttaaccttggacccattgccttagttgggattggtatttttttcggtgggtttttcatatccttatttgcatttaagttaattctgggctttagaattgctgcgaacatcttcactgatgctagagttaccatgatgagagaagtgctgaataatataaaaatgattaaatattatacgtgggaggatgcgtatgaaaaaaatattcaagatattaggaccaaagagatttctaaagttagaaaaatgcaactatcaagaaatttcttgattgctatggccatgtctttgcctagtattgcttcattggtcactttccttgcaatgtacaaagttaataaaggaggcaggcaacctggtaatatttttgcctctttatctttatttcaggtcttgagtttgcaaatgtttttcttacctattgctattggtactggaattgacatgatcattggattgggccgtttgcaaagcttattggaggctccagaagatgatccaaatcagatgattgaaatgaagccttctcctggctttgatccaaaattggctttaaaaatgacacattgctcatttgagtgggaagattatgaattaaacgacgctattgaagaagcaaaaggagaagctaaagatgaaggtaaaaagaacaaaaaaaagcgtaaggatacatggggtaagccatctgcaagtactaataaggcgaaaagattggacaatatgttgaaagacagagacggcccggaagatttagaaaaaacttcgtttaggggtttcaaggacttgaacttcgatattaaaaagggcgaatttattatgattacgggacctattggtactggtaaatcttcattattgaatgcgatggcaggatcaatgagaaaaactgatggtaaggttgaagtcaacggggacttattaatgtgtggttatccatggattcaaaatgcatctgtaagagataacatcatattcggttcaccattcaataaagaaaagtatgatgaagtagttcgtgtttgctctttgaaagctgatctggatattttaccggcaggcgatatgaccgaaattggggaacgtggtattactttatctggtggtcaaaaggcacgtatcaatttagccaggtctgtttataagaagaaggatatttatctattcgacgatgtcctaagtgctgtcgattctcgtgttggtaaacacatcatggatgaatgtctaaccggaatgcttgctaataaaaccagaattttagcaacgcatcaattgtcactgattgagagagcttctagagtcatcgttttaggtactgatggccaagtcgatattggtactgttgatgagctaaaagctcgtaatcaaactttgataaatcttttacaattctcttctcaaaattcggagaaagaggatgaagaacaggaagcggttgttgccggtgaattgggacaactaaaatatgaatcagaggtaaaggaattgactgaactgaagaaaaaggctacagaaatgtcacaaactgcaaatagtggtaaaattgtagcggatggtcatactagtagtaaagaagaaagagcagtcaatagtatcagtctgaaaatataccgtgaatacattaaagctgcagtaggtaagtggggttttatcgcactaccgttgtatgcaattttagtcgttggaaccacattctgctcacttttttcttccgtttggttatcttactggactgagaataaattcaaaaacagaccacccagtttttatatgggtctttactccttctttgtgtttgctgctttcatattcatgaatggccagttcaccatactttgcgcaatgggtattatggcatcgaaatggttaaatttgagggctgtgaaaagaattttacacactccaatgtcatacatagataccacacctttgggacgtattctgaacagattcacaaaagatacagatagcttagataatgagttaaccgaaagtttacggttgatgacatctcaatttgctaatattgtaggtgtttgcgtcatgtgtattgtttacttgccgtggtttgctatcgcaattccgtttcttttggtcatctttgttctgattgctgatcattatcagagttctggtagagaaattaaaagacttgaagctgtgcaacggtcttttgtttacaataatttaaatgaagttttgggtgggatggatacaatcaaagcataccgaagtcaggaacgatttttggcgaaatcagattttttgatcaacaagatgaatgaggcgggataccttgtagttgtcctgcaaagatgggtaggtattttccttgatatggttgctatcgcatttgcactaattattacgttattgtgtgttacgagagcctttcctatttccgcggcttcagttggtgttttgttgacttatgtattacaattgcctggtctattaaataccattttaagggcaatgactcaaacagagaatgacatgaatagtgccgaaagattggtaacatatgcaactgaactaccactagaggcatcctatagaaagcccgaaatgacacctccagagtcatggccctcaatgggcgaaataatttttgaaaatgttgattttgcctatagacctggtttacctatagttttaaaaaatcttaacttgaatatcaagagtggggaaaaaattggtatctgtggtcgtacaggtgctggtaagtccactattatgagtgccctttacaggttgaatgaattgaccgcaggtaaaattttaattgacaatgttgatataagtcagctgggacttttcgatttaagaagaaaattagccatcattccacaagatccagtattatttaggggtacgattcgcaagaacttagatccatttaatgagcgtacagatgacgaattatgggatgcattggtgagaggtggtgctatcgccaaggatgacttgccggaagtgaaattgcaaaaacctgatgaaaatggtactcatggtaaaatgcataagttccatttagatcaagcagtggaagaagagggctccaatttctccttaggtgagagacaactattagcattaacaagggcattggtccgccaatcaaaaatattgattttggatgaggctacatcctcagtggactacgaaacggatggcaaaatccaaacacgtattgttgaggaatttggagattgtacaattttgtgtattgctcacagactgaagaccattgtaaattatgatcgtattcttgttttagagaagggtgaagtcgcagaattcgatacaccatggacgttgtttagtcaagaagatagtattttcagaagcatgtgttctagatctggtattgtggaaaatgatttcgagaacagaagttaa

SEQ ID NO: 4 - DNA, 1020 bp, Saccharomyces cerevisiae
  source 1..1020: mol_type = other DNA
atgccattacctccgtcaacattgaaccagaaatctaatagagtctactctgtagctagggtgtacaagaatgcctgcgaggagagaccacaagaatactgggactacgaacaaggggtgaccatcgattggggaaagatttccaattacgaaattatcaacaaaattggaagagggaaatattccgaagtgttcagcggtagatgtattgtaaacaaccagaagtgtgttattaaagttttaaaaccagttaaaatgaaaaaaatttatagagagttgaaaattctgaccaatctaacaggcggccccaatgttgttggcctttatgatatagtacaagacgctgactccaaaatacctgctttgatctttgaggaaatcaaaaatgttgatttcagaactttatatcctacattcaaacttcctgacatccagtattatttcacgcaattattgattgcgttagactactgtcactccatgggcataatgcacagagacgtaaagcctcagaatgtcatgattgatcctacggaacgtaaactaaggctgatcgattggggcctggcggagttctaccatccaggtgtagattacaacgttcgtgtcgcttcgcgttaccacaagggaccagaacttttagtaaacttgaaccaatatgactactccctagacttatggtcagtaggatgcatgctagcagctattgtcttcaaaaaagaaccttttttcaaagggtcgtctaatccagatcaactggtaaagattgccacagtactaggaaccaaggaactgttaggctatttgggtaagtacgggttgcacttaccatctgaatacgacaacattatgagagactttacaaaaaaatcgtggacacactttataacctccgagaccaaattagctgttcctgaagtggttgatttaatcgacaatttattaaggtatgaccatcaagaaagattaacagcaaaggaggctatggatcataagtttttcaaaacgaagtttgaataa

SEQ ID NO: 5 - DNA, 1230 bp, Saccharomyces cerevisiae
  source 1..1230: mol_type = other DNA
atggtagatggactgaatacctcgaacattaggaaaagagccaggactctctctaaccccaatgactttcaagagcctaattacttgctggatcccggtaatcatccctcagatcattttcgaactcgaatgtccaaatttcggtttaatattagagagaagctgttagtgtttaccaacaatcaatcattcacattaagccgctggcaaaagaagtaccgttctgcgtttaatgatctctactttacttatacttccttaatgggatcgcataccttctatgttctgtgtttacctatgcccgtgtggtttggatattttgaaacaacaaaagatatggtttatatcttgggatattctatctacttgagtggtttttttaaagattactggtgcttgcccaggcctagagcacctccattacatcgaattacgttaagtgaatatacaacgaaggaatatggtgctccaagctcccatacagcaaatgcaacaggagtgagtctcttgtttctctacaacatctggaggatgcaagaatcttctgtcatggtccaactattgttgtcatgtgtggttttattttattatatgactttggttttcggtagaatatactgtgggatgcatggcatattagatttagtaagcggtgggctcattggaatagtgtgtttcattgttaggatgtatttcaagtacaggtttccgggtttacgcattgaggagcattggtggtttcctttgtttagtgtgggatggggtcttcttcttttgtttaaacatgttaagcccgtagacgaatgtccttgcttccaagatagtgttgcgttcatgggcgttgtgtcaggtattgaatgctgtgattggttgggcaaagtgtttggagtcaccctggtgtacaatttggaacctaactgtggctggcggttaaccttagccaggctgctggtgggcctaccgtgcgttgttatctggaagtacgtgatcagcaaaccgatgatctacacgttattgatcaaagttttccatctgaaggatgacagaaacgttgcggcaagaaaaagactggaggccacgcacaaagaaggtgcaagcaagtacgaatgtccattatatattggagagcccaagattgacattctaggtagatttattatctatgctggcgttccattcaccgttgtaatgtgcagccccgtcctattttccctcttaaatatagcataa

SEQ ID NO: 6 - DNA, 495 bp, Saccharomyces cerevisiae
  source 1..495: mol_type = other DNA
atggcagaacaaattagtcacaaaaagtccctgagggtgagcagtctgaacaaagatagaagattgctcttaagagaattctacaacttggaaaatgaaccgaataaaggtcgtcaagaagcacgtataggggaaaaagccagcgaggctcattctggggaggagcaagtcacagatgtgaatatagataccgaagcaaacactgagaaaccagtgaaagatgacgaattgagtgcaactgaagaggatctcaaggaggggtcagaagatgcagaagaagagataaagaacctgccctttaagaggctagtacaaattcacaataagctcttgggcaaagaaaccgagactaacaattctatcaagaataccatttacgaaaactattatgacctaataaaagtcaacgaccttctcaaagaaattacaaacgccaatgaagaccaaattaacaagttgaagcaaacagtagaatctttaatcaaggaactgtaa

SEQ ID NO: 7 - DNA, 1926 bp, Saccharomyces cerevisiae
  source 1..1926: mol_type = other DNA
atggatgttctcaaagaggtgttgtcactagaccaagataaatttgaccagctgaaggaaacgagccgagataaaacaaatgaaacggatgatccttttgaaaactatttgaaggattgtaaatttaaagcgccttcaaacaaagatcagtcaccatttgctaaacttaaatcattacaggaaactcattctaacaatgaagcggctattaatataattattcctcaattgattgattacttaaccgaattcactaataggttatcaaattacacacaagatttagacttcattaaaaaaaagtccaatgaattacagtcattgctcgaatacaactccactaaactggcacatatctctcctatggttaatgatttgatgattcctcctgaactcattgatgacatcattaaagggaagatcaatgaaagctggcaggataatataacattcatagcagataaagaagaaatttataacaagtataggtccaataatctcgatcaagacaacaaggacgcagaaaattcagcaatgctagcaccaaaggattttgataagttatgtcaactcctggacatcctaaaaaatgttattctagaaaggtcgaaaagacttattatttccaaaatcaaaactttgaggagtcataacccagtaccatcgcaaaggatacaaaacaaattattaaaagttcaaaaaattttccccttcataagagataataatctctccttagcccttgagttaagacaggcatattgttacacaatgaaatggtattatagagaatacttttctagatatatcaggtcattgactattttgcaatttcaacaaatcgactcgcaatttgcattgggtaatggcctttctacaacttcagtgagtgggtttaacaattcaccatcattgtttttctcaaattatctaactacatccgcttcaaatgctttctataataaactccctgtaacagatgagaaaattgataaatactttcagataaagaaaagattgaacattttaacacaagaagacaatacggtaatggtatcccaaattgcagaaaataacacaacgaaaaactacattgaaattggatttaaaaatttaaaccttgcaattttagataactgtacggtggagtaccattttttaaaagatttcttcgctatgaatggcgataattttgaggaaattaatggtttattggaacaaatatttcaaccaacttttgatgaagccacaacatacactcaacaactgatccaatataattatgacatttttggtgtattaataagtattcgtgtggccaatcaattacaatttgaatcagaaaggagaggaataccgtctatgtttgatagtttcttgaatggtcaattaattcaattatggcctcgatttcagcaattggtcgattttcaatgcgagagcttacgaaaagcggcaataactacaaatgtggcaaaatatgccggcaactcaagcacatccaatagtagccctttgacctcacctcatgagttaactgtacagttcggtaaatttttatcaagcttcttgacgttggcaataacacataagcagtccatagacgaaagatctgaacccttatacaattccatcattagattaagaaatgatttcgaaacagtcatgacaaagtgcagtaaaaagacgaaatcaccagaaagatttctggctacaaattacatgtatttatacaataacctacagcaattgcatctacatttaaatataaatgactcggatgcacaaaactacaattttgattctgctgaaaatgttggtacgaaagttgcgaatgacgacgataatgattcaagcgtaccactaataatcagagagaccgaaaatcatttcaaaactttagttgaagctttcaccagaaattga

SEQ ID NO: 8 - DNA, 2469 bp, Saccharomyces cerevisiae
  source 1..2469: mol_type = other DNA
atgctggaaggtacggtagattatgacccgctggaagatattaccaatatacttttttcaaaagaatccctgaacaacatagatgaactgatcagtattaccagaagctacaaaaagcaattgcaagaggatattctcaaagaagagaatgaattgaaggaacaccctaaaaattccgctgaaatagaggcttctctgaggaaagttttccaagatttcaaagaaactcaagatgtctcagcctccaccgagttgacgatatcgaatctgacagaaggtatctcgtacctggacattgccaagaaaaacctcacccactctttgactcttttccaaaatttaaagatattgacagacagttacatacaatgcaatgaattactctcacagggctcattcaaaaaaatggtgtccccttataagataatgtgttcgcttgctgaaaacacattcatctcttacaaatcattggacgagataaactatttgttgagctccatttcaagactgaaaggagacactttgtccaaaattaaacaaaactacaatgcgctcttttccggcggcaatatctcagagcatgatacagcactcactatggaattgcgcgaaggtgcctgcgagctactcgactgcgatacaagtacgagagcccagatgatagattggtgtttggacaaacttctcttcgaaatgaaagagatatttagggtcgacgatgaagccggatccctagaaaatttatcgagaagatacatttacttcaaaaaaattcttaataacttcaattcaaagttcgcagactatttcttaaaagactgggaaatggcagtcagattgaccaccactttttatcacattacacacaaggaccttcagacacttctgaaaagggaattcaaagacaagaacccttccattgatctattcatgacagcattacaatcgacgctagatttcgaaaaatacatcgacgtacgattttcaaaaaaaattaaggaaccaaaactaagttcctgcttcgaaccttatttgactttatgggtgtctcaccaaaaccaaatgatggaaaagaaatttctttcttatatgagtgagccgaagtacccatctaatgaaacagaatctctcgtgttaccctcgagtgcagaccttttcaggacatatcgttccgtactgactcagaccttagagctcattgataataatgccaatgatagcatattgacttcattggcaaattttttcagtagatggcttcaaacttactcacaaaaaattcttcttcctttactgctgcccgacaatattgaagtccaggataagctagaagctgccaagtataccgttttattgatcaatactgcagattattgtgccacgactatagatcaattggaggataaattatctgaattcagcggtaatcgtgaaaagctggcaaacagttttacgaaaacgaaaaatatatacgacgatttactagcaaaaggaacttcttttctattaaaccgtgtcatacccttagatctaaattttgtatggagagagtttatcaacaatgattggtcaaatgctgcgatagaagattatagcaggtacatggtaaccctcaaatccgtacttaaaatgcccgcattaacagatgcctctattaaacaacagcaagagcaaccttcgactttggcatttattttgtcgcaattcaatagagatgtttataagtggaatttcttggataaggtgattgatatcatcactacaaattttgtaagcaataccatccgccttctgcagcccgttccacccttttccctggcgggcagcaaaaggaaatttgaaaccagaactgttgtcaacattggcgagcagcttctccttgatttagaattgctgaaggagatttttcacactttaccagaaagtgtaagtaacgattctgacttgcgagaaaatacctcttataagagggtgaaaagacatgcagacaataatatagaccagctgctgaagtttattaaacttctaatggctcctctggattccgctgatgactattacgagacctactccaaattgaccaataataaccctgattcagcggtatggtcttttgtcctcgctttaaagggcattccatgggacctggcattatggaaaaagctatggagtgcctacaacttagaaacagacgacactgacgagggcagcaggccagacagtaatcgcgatcttttcatattcaagtgggacaaggtacttttgggtcaatttgaaaacaacttggcaaggatgcaagatccgaattggtcaaaatttgtgaggcaagatctgaaaatatcaccacctgttatgaagaggatagtatccacccctcaaatacaacaacaaaaagaagaacaaaaaaagcaaagtttgagtgtcaaagacttcgtttctcactcaaggttctttaacagaggcacttga

SEQ ID NO: 9 - DNA, 64 bp, synthetic construct
  misc_feature 1..64: note = Primer
  source 1..64: mol_type = other DNA
atgccattacctccgtcaacattgaaccagaaatctaatagagtctcgtacgctgcaggtcgac

SEQ ID NO: 10 - DNA, 67 bp, synthetic construct
  misc_feature 1..67: note = Primer
  source 1..67: mol_type = other DNA
ttattcaaacttcgttttgaaaaacttatgatccatagcctccttcgcataggccactagtggatct

SEQ ID NO: 11 - DNA, 24 bp, synthetic construct
  misc_feature 1..24: note = Primer
  source 1..24: mol_type = other DNA
caaagttggatatccctaatgacc

SEQ ID NO: 12 - DNA, 25 bp, synthetic construct
  misc_feature 1..25: note = Primer
  source 1..25: mol_type = other DNA
ttactcaaaaggtaaatggctctct

SEQ ID NO: 13 - DNA, 64 bp, synthetic construct
  misc_feature 1..64: note = Primer
  source 1..64: mol_type = other DNA
atgaacactaccacatctactgttatagcagcagttgccgaccagtcgtacgctgcaggtcgac

SEQ ID NO: 14 - DNA, 67 bp, synthetic construct
  misc_feature 1..67: note = Primer
  source 1..67: mol_type = other DNA
ttaagctttcctggaagagaccttggtgttagaggtcttgacacccgcataggccactagtggatct

SEQ ID NO: 15 - DNA, 21 bp, synthetic construct
  misc_feature 1..21: note = Primer
  source 1..21: mol_type = other DNA
attcaccgtcagagggatttg

SEQ ID NO: 16 - DNA, 25 bp, synthetic construct
  misc_feature 1..25: note = Primer
  source 1..25: mol_type = other DNA
gttcaagataatgcaatgtcagtca

SEQ ID NO: 17 - DNA, 64 bp, synthetic construct
  misc_feature 1..64: note = Primer
  source 1..64: mol_type = other DNA
atggtagatggactgaatacctcgaacattaggaaaagagccaggtcgtacgctgcaggtcgac

SEQ ID NO: 18 - DNA, 67 bp, synthetic construct
  misc_feature 1..67: note = Primer
  source 1..67: mol_type = other DNA
ttatgctatatttaagagggaaaataggacggggctgcacattaccgcataggccactagtggatct

SEQ ID NO: 19 - DNA, 22 bp, synthetic construct
  misc_feature 1..22: note = Primer
  source 1..22: mol_type = other DNA
aagcctacgttttggactctca

SEQ ID NO: 20 - DNA, 20 bp, synthetic construct
  misc_feature 1..20: note = Primer
  source 1..20: mol_type = other DNA
catggccagcactattttca

SEQ ID NO: 21 - DNA, 23 bp, synthetic construct
  misc_feature 1..23: note = Primer
  source 1..23: mol_type = other DNA
gcgtttaccgtatcgcagaatgg

SEQ ID NO: 22 - DNA, 26 bp, synthetic construct
  misc_feature 1..26: note = Primer
  source 1..26: mol_type = other DNA
atggcccaatcacaaccacatcttag

SEQ ID NO: 23 - DNA, 24 bp, synthetic construct
  misc_feature 1..24: note = Primer
  source 1..24: mol_type = other DNA
atgctatcgccaaggctgtcaagg

SEQ ID NO: 24 - DNA, 30 bp, synthetic construct
  misc_feature 1..30: note = Primer
  source 1..30: mol_type = other DNA
gacaccttcatcacctaatttctcttcaac

SEQ ID NO: 25 - DNA, 64 bp, synthetic construct
  misc_feature 1..64: note = Primer
  source 1..64: mol_type = other DNA
gcgtatttgcggtgagacggaatctgacgaggatattaagtacagcagctgaagcttcgtacgc

SEQ ID NO: 26 - DNA, 67 bp, synthetic construct
  misc_feature 1..67: note = Primer
  source 1..67: mol_type = other DNA
tctcgaaggaagtgttcggtgaaagccacgatatgccgctggaaagcataggccactagtggatctg

SEQ ID NO: 27 - DNA, 20 bp, synthetic construct
  misc_feature 1..20: note = Primer
  source 1..20: mol_type = other DNA
ataggtgaggctctgcatag

SEQ ID NO: 28 - DNA, 23 bp, synthetic construct
  misc_feature 1..23: note = Primer
  source 1..23: mol_type = other DNA
cgaaggaagtgttcggtgaaagc

SEQ ID NO: 29 - DNA, 25 bp, synthetic construct
  misc_feature 1..25: note = Primer
  source 1..25: mol_type = other DNA
gttagaaatgtcttggatgcaggtg

SEQ ID NO: 30 - DNA, 23 bp, synthetic construct
  misc_feature 1..23: note = Primer
  source 1..23: mol_type = other DNA
cagcaatggcattcaagacctta

SEQ ID NO: 31 - DNA, 55 bp, synthetic construct
  misc_feature 1..55: note = Primer
  source 1..55: mol_type = other DNA
tcccaacgaccgaaaacctgtattttcagggatccatgaaagaacaccagtgtag

SEQ ID NO: 32 - DNA, 58 bp, synthetic construct
  misc_feature 1..58: note = Primer
  source 1..58: mol_type = other DNA
tcccaacgaccgaaaacctgtattttcagggatccatggctcaaactgacgacccaaa

SEQ ID NO: 33 - DNA, 55 bp, synthetic construct
  misc_feature 1..55: note = Primer
  source 1..55: mol_type = other DNA
gtcttcaggagcgagttctggctggcttgcacgtgttacttggatggttgacaag

SEQ ID NO: 34 - DNA, 20 bp, synthetic construct
  misc_feature 1..20: note = Primer
  source 1..20: mol_type = other DNA
ggttacgctattggtgcatg

SEQ ID NO: 35 - DNA, 19 bp, synthetic construct
  misc_feature 1..19: note = Primer
  source 1..19: mol_type = other DNA
gcaaggaaagtgaccaatg

(19) United States
(12) Patent Application Publication
(10) Pub. No.: US 2026/0152779 A1
(43) Pub. Date: Jun. 4, 2026

(54) GENETICALLY ENGINEERED FUMONISIN SENSITIVE YEAST

(71) Applicant: FFOQSI GMBH, Tulln (AT)

(72) Inventors: Rudolf MITTERBAUER, Scheffau am Wilden Kaiser (AT); Gerhard ADAM, Tulln (AT); Tamara KRSKA, Tulln (AT); Krisztian TWARUSCHEK, Tulln (AT); Franz BERTHILLER, Tulln (AT)

(73) Assignee: FFOQSI GMBH, Tulln (AT)

(21) Appl. No.: 18/864,326

(22) PCT Filed: May 15, 2023
(86) PCT No.: PCT/EP2023/062973

§ 371 Date: Nov. 8, 2024

(30) Foreign Application Priority Data
May 16, 2022 (EP) 22173497.3

(51) Int. Cl. C12Q 1/18; C12N 1/18; C12N 9/10; C12N 9/16; C12Q 1/02; C12R 1/865

(52) U.S. Cl. CPC C12Q 1/18; C12N 1/18; C12N 9/1029; C12N 9/16; C12Q 1/025; C12R 2001/865; C12Y 203/01; C12Y 301/01087

(57) ABSTRACT

The invention refers to a fumonisin-sensitive yeast, specifically Saccharomycescerevisiae, comprising disrupted or deleted genes SNQ2, PDR12, YOR1, CKA2, LCB3, and optionally any one of VPS51, VPS52, or VPS53 and its use for detecting inhibitory activity of fumonisins or detecting fumonisin detoxifying compounds in a sample. The fumonisin-sensitive yeast is also useful for screening of fumonisin detoxification or resistance conferring activity of heterologous polypeptides.

### FIELD OF THE INVENTION

The invention refers to genetically engineered fumonisin-sensitive yeast for high-throughput monitoring of fumonisin toxicity and can serve for detection and expressing fumonisin detoxifying or resistance conferring compounds.

### BACKGROUND OF THE INVENTION

Fumonisins are a class of toxicologically relevant Fusarium secondary metabolites. Maximum tolerated levels of these mycotoxins in food commodities and guidance levels for feed were enacted in the EU after hazard characterization (Opinion of the Scientific Committee on food on Fusarium toxins Part 3: Fumonisin B₁ (FB₁), European Commission, SCF/CS/CNTM/MYC/24) and risk assessment. Fumonisins are produced by Aspergillus and Fusarium species, such Fusarium verticillioides, F. proliferatum and other species that are mainly a problem of corn production in warmer climates, particularly under drought conditions (Southern USA, Spain, Italy). Yet, due to climate change, fumonisin producing Fusarium strains are becoming increasingly important also in central Europe, including Austria. Fumonisins are hepatotoxic and carcinogenic in animals (Gelderblom W C et al, 1991, Gelderblom W C et al, 1992; Voss K A et al, 2002) and cause equine leukoencephalomalacia (Marasas W F et al, 1988) and porcine pulmonary edema (Harrison L R et al, 1990). Consumption of fumonisin-contaminated food is correlated with neural tube defects (Missmer S A et al, 2006) and esophageal cancer in humans.

Fumonisins are a family of reduced linear polyketides that contain two tricarballylic ester groups and a primary amine derived typically from the condensation of L-alanine with the polyketide backbone. Fumonisin B₁ (FB₁) is the most relevant (abundantly formed) metabolite of this class, while FB₂, FB₃, FB₄, and FB₆ are also widespread and differ solely in the number and position of hydroxyl groups along the polyketide backbone.

The search for microbes capable of fumonisin degradation or for enzymes detoxifying fumonisins currently relies primarily on chemical detection methods. Fumonisins are structurally similar to sphingolipids and act as inhibitors of eukaryotic sphingolipid biosynthesis, by specifically targeting ceramide synthases of animals (Merrill A H Jr et al., 1996), plants (Luttgeharm K D et al, 2016), and also fungi. Although still controversial, there is increasing evidence that fumonisin production is a virulence factor of Fusarium species on different host plants (Sun L, 2019, Glenn A E et al, 2008). Inhibition of ceramide synthase leads to imbalances of membrane lipids that can trigger programmed cell death in plants and animal cells (Sun L et al, 2019). Enzymatic detoxification of fumonisin is an attractive method to up-cycle fumonisin-contaminated feed commodities. Furthermore, preventing accumulation of toxins in the field by expressing a heterologous fumonisin-detoxification gene or an insensitive target in genetically engineered crop plants might also be an attractive strategy to reduce fungal virulence and diminish mycotoxin levels. The search for useful genes is ongoing.

Enzymatic modification of fumonisins is an attractive method to mitigate their toxicity. Fumonisin degrading enzymes have been identified in microorganisms that metabolize fumonisins as an energy source, but not in species that synthesize fumonisins. Previously known wild-type enzymes isolated from native source (bacterial or fungal) that target the amine functional group of fumonisins require hydrolyzed fumonisins as substrates (ie: fumonisins lacking the tricarballylic ester moieties). This necessitates prior deesterification via an additional enzyme that complicates the detoxification process. The aminotransferase Fuml requires pyruvate as co-substrate and pyridoxal phosphate as co enzyme (Hartinger D et al, 2011). These requirements limit the usefulness of Fuml as a fumonisin detoxification enzyme due to the expense of the cofactors and added complexity of the system.

The monitoring of the biological action of fumonisins is difficult. Highly purified toxin preparations are needed with mammalian cells cultured in vitro, to avoid unspecific toxic effects of other metabolites co-occurring in crude toxin extracts. The reported susceptibility of different human and animal cell lines are summarized by Molina-Pintor I B et al, 2021. Depending on the cell type and length of incubation cytotoxic effects are typically observed in the 10-100 UM range. Reported acute cytotoxicity (IC₅₀ values) for FB₁ for primary rat hepatocytes are 2000 UM (Gelderblom W C et al, 1993), while human HepG2 cells are more sensitive (IC₅₀ 399 UM) (Mckean C et al, 2006). Plants are typically more sensitive. For example, Arabidopsis seed germination and root development is already strongly inhibited at 1 μM FB₁ (Stone J M et al, 2000) but such bioassays are quite time consuming (e.g. 10 days).

Recently a plant bioassay based on duckweed (Lemna minor) was used to characterize the toxicity and structure/activity relationship of different fumonisin metabolites (Renaud J B et al, 2021) This method is based on counting green pixels (Lemna leaf surface) in 24 well plates during 5 days of growth. Other similarly labor-intensive and low-throughput whole-animal test systems were described. For instance, Hydra attenuata was used to monitor toxicity by microscopic observation of FB₁-induced death/disintegration (Lemke S L, 2001) after up to 96 hours incubation. Similarly, microscopic observation of non-motile/dead brine shrimp (Artemia salina) was used to determine toxicity after 24 h incubation (Hartl M and Humpf H U, 2000). Depending on the incubation time IC50 values for FB₁ for brine shrimp ranging from 1.7 μM (48 h) to about 10 μM after 24 h were reported (Hlywka J J et al, 1997).

Burgess K. M. N. et al. (2016) describe mechanistic insights into the biosynthesis and detoxification of fumonisin mycotoxins. Thereby, the toxicity of fumonisins were tested using a Lemna minor bioassay.

Abbas H K et al., (1993) describe the detection of toxicity of fumonisins using a jimsonweed (Datura stramonium L.)-based and a mammalian cell culture-based bioassay.

Abolmaali S. et al., (2008) describe the use of Saccharomyces cerevisiae as bioassay organism for detecting fungal toxins.

Rogers B at al., (2001) describe the pleiotropic drug ABC transporters from Saccharomyces cerevisiae.

Miyake R. et al., (2022) describe a genetic biosensor for the detection and production of short-branched chain fatty acids in Saccharomyces cerevisiae.

Kobayashi S D. and Nagiec M M, (2003) describe the regulation of ceramide synthesis by Elo3p and Cka2p in Saccharomyces cerevisiae.

Qie L. et al., (1997) describe the identification of a Saccharomyces gene, LCB3, and its requirement for incorporation of exogenous long chain bases into sphingolipids.

Takagi K. et al., (2012) describe the involvement of a golgi-associated retrograde protein complex in the recycling of the putative Dnf aminophospholipid flippases in yeast.

None of these test organisms, however, can serve as convenient host for monitoring toxicity of fumonisins and for testing the function of candidate detoxification genes by heterologous expression.

There is thus a clear and yet unmet need in the field for improved bioassay strains for high throughput monitoring of toxicity of fumonisins and for detecting detoxifying or resistance conferring compounds.

### SUMMARY OF THE INVENTION

It is the objective of the present invention to provide fumonisin-sensitive indicator organisms for detecting of fumonisins and high throughput monitoring of toxicity of fumonisins, which can also serve as host to phenotypically detect and evaluate expressed detoxification or resistance conferring heterologous polypeptides.

The objective is solved by the subject of the claims and as further described herein.

It was surprisingly found in the present invention that specific modifications introduced into a yeast strain increase the sensitivity of said yeast to fumonisin. Said combination of modifications allowed the genetically engineered yeast to sufficiently grow under common cultivation conditions while being highly sensitive towards fumonisin, specifically FB₁.

The present invention provides a modified fumonisin-sensitive yeast comprising disrupted or deleted genes SNQ2, PDR12, YOR1, CKA2, and LCB3, and optionally any one of VPS51, VPS52, or VPS53.

Specifically, all of genes SNQ2, PDR12, YOR1, CKA2, and LCB3 are disrupted or deleted in the modified fumonisin-sensitive yeast described herein.

Due to these selected modifications, active efflux is diminished, activity of the toxin target ceramide synthase is reduced by interfering with posttranslational modification (phosphorylation), and uptake of phosphorylated sphingobases from the medium and intracellular recycling are targeted.

In general, the disrupted or deleted genes as described herein are highly conserved in ascomycete and also basidiomycete yeasts. Thus, a modified yeast comprising disrupted or deleted genes SNQ2, PDR12, YOR1, CKA2, LCB3, and optionally any one of VPS51, VPS52, or VPS53, is a fumonisin-sensitive yeast.

According to a specific embodiment of the invention, said yeast comprises disrupted or deleted genes having a sequence identity of at least 50% with any one of SEQ ID NOs: 1, 2, 3, 4, and 5. Specifically, said yeast comprises disrupted or deleted genes having a sequence identity of 50, 55, 60, 65, 70, 75, 80, 85, 90, or 95% with any one of SEQ ID NOs: 1, 2, 3, 4, and 5.

According to a specific embodiment of the invention, the yeast is of the phylum Ascomycota, preferably a Saccharomyces cerevisiae strain.

According to a specific embodiment, the fumonisin-sensitive yeast of the invention comprises selectable marker genes, preferably resistance or auxotrophic markers, specifically selected from the group consisting of LEU2, URA3, LYS2, ADE2, TRP1, and HIS3.

Specifically, the genes SNQ2, PDR12, YOR1, CKA2, LCB3, and VPS51 are inactivated, either disrupted or deleted in the fumonisin-sensitive yeast of the invention.

More specifically, the inventive yeast strain is a Saccharomyces cerevisiae mutant strain comprising the genotype snq2::hisG pdr12::hisG yor1::hisG cka2Δ::loxP lcb3Δ::loxP vps51Δ::loxP.

According to the invention, the fumonisin is Fumonisin B₁ (FB₁), FB₂, FB₃, FB₄, FB₅, FB₆, FC₁, FC₂, FC₃, FC₄, or isomers thereof or any combination thereof.

According to one embodiment of the invention, the fumonisin is Fumonisin B₁ (FB₁), FB₂, FB₃, FB₄, FB₅, FB₆, or isomers thereof or any combination thereof.

The present invention further provides the use of the fumonisin-sensitive yeast for screening fumonisin detoxifying compounds or enzymes.

Further provided herein is the use of the fumonisin-sensitive yeast described herein for toxicological studies.

The present invention further provides a method for detecting inhibitory activity of fumonisins in a sample, comprising the steps of
i. contacting the fumonisin-sensitive yeast of the invention with the sample;
ii. determining the growth of said fumonisin-sensitive yeast in the presence and absence of said sample; wherein
iii. reduced growth indicates the presence of fumonisin in the sample.

Specifically, the sample is a plant extract, more preferably corn extract, or extract of fungal cultures.

According to an alternative embodiment of the present invention, herein provided is a method for detecting fumonisin detoxifying compounds in a sample, comprising the steps of
i. contacting the fumonisin-sensitive yeast of the invention with the sample in the presence of fumonisin;
ii. determining growth of said yeast in the presence and absence of said sample; wherein
iii. increased growth indicates the presence of fumonisin detoxification compounds in the sample.

According to a specific embodiment, the sample is an extract of fungi, crude protein preparation, or purified protein.

According to a further embodiment, the fumonisin detoxifying compounds are polypeptides, proteins, preferably the compounds are enzymes.

According to a further embodiment, herein provided is also a method for screening the fumonisin detoxification or resistance conferring activity of a heterologous polypeptide, comprising the steps of
i. introducing a heterologous DNA sequence encoding a potential fumonisin detoxification or resistance conferring polypeptide into the fumonisin-sensitive yeast of the present invention;
ii. expressing the potential fumonisin detoxification or resistance conferring polypeptide in said yeast;
iii. determining the growth of said yeast;
iv. whereby growth of said yeast in the presence of fumonisin compared to a reference yeast lacking said heterologous DNA sequence indicates detoxification or resistance conferring activity of the polypeptide.

Specifically, the fumonisin detoxification or resistance conferring polypeptide is an enzyme, an oxidase, preferably an amine oxidase, an N-acetyltransferase, or an esterase.

In a specific embodiment, the detoxification or resistance conferring activity of one or more polypeptides is screened, wherein the one or more heterologous DNA sequence encoding said one or more polypeptides are introduced into the same fumonisin-sensitive cell or in separate fumonisin-sensitive yeasts.

### FIGURES

FIG. 1: Schematic disclosure of the sphingolipid biosynthesis pathway in yeast (indicating the affected steps).

FIG. 2: Fumonisin B₁ sensitivity of YPH500 (control strain) and YRU74 (lacking ABC transporters: snq2 pdr12 yor1 mutant) and YRU94ML (snq2 pdr12 yor1 cka2Δ lcb3Δ) and YTKT33 (snq2 pdr12 yor1 cka2Δ lcb3Δ vps51Δ) in YPD medium with increasing concentrations of crude FB₁.

FIG. 3: Fumonisin B₁ sensitivity of YPH500 (“wild-type”) and YRU74 (snq2 pdr12 yor1) and YRU94ML (snq2 pdr12 yor1 cka2Δ lcb3Δ) and YTKT33 (snq2 pdr12 yor1 cka2Δ lcb3Δ vps51Δ) on YPD agar plates with increasing concentrations of crude FB₁.

FIG. 4: Fumonisin B₁ sensitivity of YPH500 (“wild-type”) and YRU74 (snq2 pdr12 yor1) and YRU94ML (snq2 pdr12 yor1 cka2Δ lcb3Δ) and YTKT33 (snq2 pdr12 yor1 cka2Δ lcb3Δ vps51Δ) in SC medium with increasing concentrations of crude FB₁.

FIG. 5: Fumonisin B₁ sensitivity of YPH500 (“wild-type”) and YRU74 (snq2 pdr12 yor1) and YRU94ML (snq2 pdr12 yor1 cka2Δ lcb3Δ) and YTKT33 (snq2 pdr12 yor1 cka2Δ lcb3Δ vps51Δ) on SC-URA agar plates (lacking uracil) with increasing concentrations of crude FB₁.

FIG. 6: Fumonisin B₁ sensitivities of the candidate genes YOR1, FvCER1, AspAmOx (Fumonisin amine oxidase from Aspergillus), and FumD esterase.

FIG. 7: Strain YTKT33 was mixed into SC-agarose medium (42° C.), and paper disks were placed on the solidified medium. Upper raw: Left-water control. Right-Crude extract of F. verticillium from autoclaved maize was added (giving a inhibition zone) after 3 days incubation. Lower row: Left-Fumonisin degrading Fumzyme was added, diffusing into the inhibition zone from fumonisin added on the right (leading to the distorted shape of the halo).

FIG. 8: Results of testing sensitivity of YTKT33 to different B-type fumonisins. The sensitive yeast strain YTKT33 was grown in SC medium and exposed to different concentrations of FB1, FB2, FB3 and FB4. Strain inoculum was pipetted into a microtiter well plate with 0.6N dilutions of the respective fumonisins. After 24 h at 30° C. the optical density at 600 nm (OD600) was measured to monitor growth. The blank (medium without yeast) was subtracted from the measured OD600 values. On the x-axis, the graphs show the concentration of the respective fumonisins in μM (log 2 scaled). Means and standard deviations were calculated from 4 replicates.

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017); Berg et al, “Stryer Biochemie” Springer Verlag, 2018; and Murphy & Weaver, “Janeway's Immunobiology” (9th Ed., or more recent editions), Taylor & Francis Inc, 2017, Guthrie C and Fink G R, Guide to Yeast Genetics and Molecular and Cell Biology, Part B, 350, 3-623 (2002).

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, expression constructs, transformed host cells and modified proteins and enzymes, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

The method for yeast transformation is not particularly limited, and methods conventionally used for transformation can be used. Yeast transformation can be performed in accordance with the lithium acetate method as described by Rothstein R, 1991. Examples of further methods include protoplast method, KU method (treatment of yeast cells with alkali metal ions), KUR method (simplified KU method, omission of the heat-shock process), electroporation method, and a method using a carrier DNA (Gietz R D et al, 1995). Yeast-genetic methods, especially for Saccharomyces cerevisiae, are in accordance with the methods described in Sherman F, 1981, which comprises the crossing of the modified strains and isolation of the diploid strains by micromanipulation. The integration is preferably followed by means of selectable markers (auxotrophy and/or resistances). In detail, cassettes containing markers are introduced, the subsequent crossing results in isogenic strains being obtained and selection of those strains which have a stable integration of the desired cassettes in the yeast genome after transformation, growth in culture media and selection of the strains.

The terms “deletion” and “disruption”, refer to the elimination of the entire coding region of the gene, or disruption of the coding region by introducing heterologous sequences, or modification of the respective promoter and/or terminator region such as by deletion, insertion or mutation so that the gene either does not express the protein or an active version of the protein, or produces an enzyme with significantly reduced activity. Such methods are well known by the skilled person. The deletion or disruption can be accomplished by genetic engineering methods, forced evolution, or mutagenesis, followed by appropriate selection or screening to identify the desired mutants.

Disruption of a gene can be attained by, for example, deleting a part or the whole of the coding region of the gene on a chromosome. Furthermore, the whole of a gene including sequences upstream and downstream from the gene on a chromosome may be deleted. The region to be deleted may be any region such as an N-terminus region, an internal region, or a C-terminus region, so long as the activity of the protein can be reduced. Deletion of a longer region can usually more surely inactivate the gene. Further, it is preferred that reading frames of the sequences upstream and downstream from the region to be deleted are not the same.

Disruption of a gene can also be attained by, for example, introducing a mutation for an amino acid substitution (missense mutation), a stop codon (nonsense mutation), a frame shift mutation which adds or deletes one or two nucleotide residues, or the like into the coding region of the gene on a chromosome.

Disruption of a gene can also be attained by, for example, inserting another sequence into a coding region of the gene on a chromosome. Site of the insertion may be in any region of the gene, and insertion of a longer region can usually more surely inactivate the gene. It is preferred that reading frames of the sequences upstream and downstream from the insertion site are not the same. The other sequence is not particularly limited so long as a sequence that reduces or eliminates the activity of the encoded protein is chosen, and examples thereof include, for example, a marker gene such as antibiotic resistance genes, and a gene useful for production of an objective substance.

According to one embodiment, both disruption and deletion of a gene leads to loss of function of this gene and thus, the gene either does not express the protein or an active version of the protein, or produces an enzyme with significantly reduced activity.

To obtain the fumonisin-sensitive yeast strain of the invention, the SNQ2, PDR12, YOR1, CKA2, LCB3, and optionally the VPS51, VPS52, or VPS53 target genes can be deleted and/or disrupted by e.g. introducing one or more selectable markers (auxotrophy and/or resistances). The below listed sequences are from strain S288C, however the respective sequences can also be derived from any other yeast strain such as, but not limited to CEN.PK, D273-10B, FL100, JK9-3d, SEY6210, Sigma1278b, SK1, W303, X2180-1A, Y55.

The sequences of the target genes are as follows:

```
SNQ2 (YDR011W), sequence from strain S288C:
(SEQ ID NO: 1)
      1 ATGAGCAATA TCAAAAGCAC GCAAGATAGC TCTCATAATG CTGTCGCTAG AAGCTCAAGC
    61 GCTTCTTTTG CAGCTTCAGA AGAATCATTT ACGGGCATAA CCCATGACAA AGATGAGCAG
  121 AGCGATACCC CGGCGGATAA ACTAACAAAA ATGCTGACAG GACCTGCAAG AGACACTGCG
  181 AGCCAGATTA GTGCCACTGT GTCTGAAATG GCGCCAGATG TCGTATCTAA AGTGGAGTCA
  241 TTTGCAGATG CACTATCCCG TCATACAACG AGAAGCGGTG CCTTTAATAT GGATTCAGAT
  301 AGTGACGATG GGTTCGATGC CCATGCCATC TTTGAAAGTT TTGTAAGAGA CGCTGATGAG
  361 CAAGGCATCC ATATCCGCAA GGCTGGTGTT ACCATAGAGG ACGTAAGCGC TAAAGGTGTG
  421 GATGCGAGTG CCCTAGAAGG TGCTACCTTT GGTAACATTC TTTGTTTACC GTTGACCATC
  481 TTTAAAGGTA TTAAGGCTAA GAGGCATCAA AAGATGAGAC AGATCATAAG CAATGTCAAT
  541 GCCCTGGCAG AAGCGGGTGA AATGATTTTG GTTCTTGGAA GGCCTGGTGC TGGTTGTTCC
  601 TCCTTTTTAA AAGTAACAGC TGGTGAAATA GATCAGTTTG CCGGTGGTGT TTCCGGTGAA
  661 GTAGCATATG ATGGTATTCC CCAAGAAGAA ATGATGAAAC GATATAAAGC AGATGTTATT
  721 TACAATGGTG AGTTGGATGT TCATTTCCCT TATTTAACAG TTAAGCAAAC TTTGGATTTC
  781 GCTATTGCCT GCAAAACGCC TGCTCTCAGA GTCAATAACG TTTCCAAAAA GGAATACATT
  841 GCATCCAGAA GAGATTTATA TGCAACCATT TTCGGTCTAA GGCATACCTA TAATACCAAA
  901 GTTGGTAACG ATTTCGTTAG AGGTGTATCT GGTGGTGAAC GTAAGCGTGT TTCCATTGCC
  961 GAGGCTTTGG CAGCCAAAGG TTCCATTTAC TGTTGGGATA ATGCCACTAG AGGTTTGGAT
1021 GCGTCTACGG CCTTAGAATA CGCAAAAGCC ATCCGTATTA TGACAAACTT ATTGAAATCA
1081 ACCGCTTTTG TTACAATTTA TCAGGCAAGT GAAAACATTT ACGAAACATT TGATAAAGTC
1141 ACTGTCCTTT ATTCTGGTAA GCAAATTTAT TTTGGTTTGA TCCACGAGGC AAAACCTTAT
1201 TTCGCAAAAA TGGGTTATTT GTGTCCTCCA AGGCAAGCAA CAGCTGAATT TTTAACCGCG
1261 TTGACTGATC CAAATGGATT CCATCTGATC AAGCCAGGTT ATGAAAATAA AGTACCAAGA
1321 ACCGCTGAGG AATTCGAAAC ATATTGGTTA AATTCTCCAG AGTTTGCTCA AATGAAAAAA
1381 GATATCGCTG CTTATAAAGA GAAGGTCAAT ACCGAAAAGA CTAAAGAAGT TTATGACGAA
1441 TCGATGGCTC AAGAGAAATC CAAATATACG AGAAAGAAGT CTTATTATAC AGTGTCATAT
1501 TGGGAACAAG TTAAACTGTG TACCCAACGT GGGTTCCAAA GAATTTACGG TAACAAGAGT
1561 TATACAGTCA TCAATGTCTG CTCTGCAATA ATTCAATCTT TTATTACTGG ATCATTATTT
1621 TACAATACCC CTTCATCCAC TTCCGGTGCT TTTTCAAGAG GTGGTGTGTT GTATTTTGCG
1681 CTACTATATT ATTCTTTGAT GGGACTGGCG AATATTTCTT TTGAACATAG GCCAATCTTA
1741 CAAAAGCACA AGGGCTATTC TTTGTATCAT CCTTCAGCTG AGGCAATTGG CTCCACTCTG
1801 GCATCTTTCC CCTTCAGAAT GATTGGTTTG ACCTGTTTCT TTATCATTTT ATTCTTCCTA
1861 TCTGGGTTGC ACAGAACAGC GGGATCATTT TTTACCATCT ATTTGTTCTT AACCATGTGT
1921 TCAGAGGCGA TCAATGGTTT ATTTGAGATG GTTTCTTCAG TATGTGACAC TCTTTCTCAA
1981 GCTAACTCTA TCTCGGGTAT TCTGATGATG TCTATCTCAA TGTACTCTAC CTATATGATC
2041 CAATTGCCTT CGATGCATCC ATGGTTTAAA TGGATATCGT ACGTACTACC TATCAGGTAC
2101 GCCTTCGAGT CGATGTTAAA TGCCGAATTT CACGGTAGGC ATATGGATTG TGCTAACACT
2161 CTAGTACCCA GTGGAGGAGA CTATGATAAT TTATCCGATG ACTACAAAGT ATGTGCTTTT
2221 GTTGGTTCGA AACCAGGTCA GTCTTATGTG CTTGGTGATG ACTACCTTAA AAATCAATTT
2281 CAGTACGTTT ATAAGCACAC GTGGAGAAAC TTTGGTATCT TGTGGTGCTT TTTACTGGGT
2341 TATGTTGTTT TGAAAGTGAT ATTCACAGAA TATAAGAGGC CTGTGAAAGG TGGTGGTGAT
2401 GCTCTTATCT TCAAGAAAGG ATCAAAAAGA TTTATCGCAC ATGCAGATGA AGAATCTCCA
2461 GACAATGTCA ATGATATAGA TGCCAAAGAG CAATTCTCCA GTGAAAGTAG CGGCGCAAAT
2521 GATGAAGTAT TTGATGATTT AGAAGCCAAA GGTGTTTTCA TTTGGAAGGA CGTATGCTTT
2581 ACTATTCCAT ATGAAGGCGG TAAGAGAATG CTTTTGGATA ATGTTTCAGG TTATTGTATT
2641 CCAGGTACCA TGACGGCCTT GATGGGAGAG TCAGGTGCTG GTAAAACAAC TTTGTTAAAT
2701 ACTCTTGCTC AAAGAAATGT CGGTATCATT ACTGGTGATA TGCTTGTCAA TGGACGTCCC
2761 ATTGATGCGA GTTTCGAAAG GCGTACAGGT TATGTACAAC AACAGGATAT ACATATCGCA
2821 GAGTTAACTG TTAGGGAATC GTTGCAGTTT TCTGCTCGTA TGCGTCGCCC TCAGCATTTG
2881 CCTGATTCTG AAAAAATGGA TTATGTGGAA AAAATCATCA GAGTTTTGGG AATGGAAGAG
2941 TATGCGGAAG CCCTTGTTGG TGAGGTTGGT TGTGGTTTAA ACGTTGAACA GAGAAAGAAG
3001 CTGTCTATTG GTGTTGAACT AGTCGCCAAA CCAGACTTAT TATTATTCCT CGATGAACCT
3061 ACATCAGGTT TGGATTCTCA ATCTTCATGG GCCATTATTC AATTATTAAG AAAGTTATCA
3121 AAAGCTGGCC AATCCATTCT TTGTACGATC CATCAACCTT CAGCTACTCT GTTCGAAGAG
3181 TTTGATAGAT TACTACTTTT GAGGAAGGGT GGACAAACTG TTTATTTCGG AGATATTGGT
3241 AAGAACTCTG CCACCATTTT GAACTACTTT GAAAGGAATG GGGCAAGAAA ATGTGATTCT
3301 AGTGAAAATC CTGCTGAATA TATTTTAGAG GCTATTGGTG CCGGTGCCAC AGCATCCGTC
3361 AAAGAAGACT GGCACGAAAA ATGGTTGAAC TCTGTCGAGT TTGAACAAAC AAAAGAAAAA
3421 GTACAGGATT TAATAAATGA TTTATCGAAA CAAGAAACTA AATCCGAAGT TGGAGACAAA
3481 CCTTCCAAAT ATGCTACTTC TTATGCTTAC CAGTTCAGAT ATGTTTTAAT CAGAACCTCT
3541 ACTTCATTTT GGAGAAGTCT GAATTACATC ATGTCAAAGA TGATGCTAAT GCTGGTTGGT
3601 GGTCTGTATA TTGGTTTCAC ATTTTTCAAT GTTGGTAAAA GTTATGTCGG CTTACAAAAT
3661 GCGATGTTCG CGGCATTTAT CTCTATTATC TTGTCTGCTC CTGCAATGAA CCAAATCCAA
3721 GGACGTGCTA TTGCCTCCAG AGAACTTTTT GAAGTTAGGG AATCCCAATC TAACATGTTT
3781 CACTGGTCGC TGGTGTTGAT CACTCAGTAC TTGAGCGAAC TTCCCTATCA TTTATTTTTT
3841 TCGACAATTT TCTTTGTCTC ATCGTATTTT CCATTAAGAA TCTTCTTCGA AGCGTCAAGA
3901 TCTGCGGTGT ACTTTTTGAA TTACTGCATT ATGTTCCAGT TATACTATGT TGGTCTTGGC
3961 TTAATGATCC TATATATGTC ACCGAACCTT CCATCCGCTA ATGTTATCTT AGGTTTGTGT
4021 CTGTCATTTA TGCTTTCTTT CTGTGGTGTT ACACAACCTG TCTCATTGAT GCCTGGCTTC
4081 TGGACATTCA TGTGGAAGGC TTCCCCATAC ACATATTTTG TTCAGAATCT GGTCGGAATT
4141 ATGCTGCACA AAAAACCAGT CGTATGCAAA AAGAAAGAAC TAAACTACTT CAACCCACCA
4201 AACGGCTCAA CGTGTGGAGA GTACATGAAA CCCTTTTTGG AAAAAGCTAC TGGTTACATC
4261 GAAAATCCTG ATGCTACGTC AGATTGTGCA TACTGTATTT ACGAAGTTGG AGATAATTAT
4321 TTGACACATA TCAGCTCTAA GTATAGCTAC TTGTGGAGAA ATTTTGGAAT ATTTTGGATT
4381 TACATTTTCT TCAATATCAT TGCTATGGTT TGTGTGTATT ACCTCTTCCA TGTAAGACAA
4441 TCTTCCTTCC TAAGCCCCGT ATCTATACTC AATAAAATTA AAAACATAAG GAAAAAGAAG
4501 CAGTAA
PDR12 (sequence from strain S288C):
(SEQ ID NO: 2)
      1 ATGTCTTCGA CTGACGAACA TATTGAGAAA GACATTTCGT CGAGATCGAA CCATGACGAT
    61 GATTATGCTA ATTCGGTACA ATCCTACGCT GCCTCCGAAG GCCAAGTTGA TAATGAGGAT
  121 TTGGCAGCCA CTTCTCAGCT ATCCCGTCAC CTTTCAAACA TTCTTTCCAA TGAAGAAGGT
  181 ATTGAAAGGT TGGAGTCTAT GGCGAGAGTC ATTTCACATA AGACAAAGAA GGAAATGGAC
  241 TCTTTTGAAA TTAATGACTT AGATTTTGAT TTGCGCTCAC TATTACATTA TTTGAGGTCT
  301 CGTCAATTGG AACAGGGAAT TGAACCTGGT GATTCTGGTA TTGCCTTTAA AAACCTAACA
  361 GCAGTCGGTG TTGATGCCTC TGCTGCATAT GGGCCTAGTG TTGAAGAGAT GTTTAGAAAT
  421 ATTGCTAGTA TACCGGCACA TCTCATAAGT AAATTTACCA AGAAATCTGA TGTCCCATTA
  481 AGGAATATTA TTCAAAATTG TACGGGTGTC GTTGAATCTG GTGAAATGTT ATTTGTCGTC
  541 GGTAGGCCAG GTGCAGGTTG CTCCACTTTC CTAAAGTGTC TATCTGGTGA AACTTCAGAA
  601 TTAGTTGATG TACAAGGTGA ATTCTCCTAT GATGGTCTGG ACCAAAGCGA AATGATGTCT
  661 AAGTATAAAG GTTACGTTAT TTACTGTCCC GAGCTTGATT TCCATTTCCC AAAAATTACT
  721 GTGAAGGAAA CAATCGATTT TGCCCTAAAA TGTAAGACTC CTCGTGTTAG AATTGACAAA
  781 ATGACGAGAA AGCAATACGT TGATAACATC AGAGATATGT GGTGTACCGT TTTTGGTTTA
  841 AGACACACAT ATGCCACCAA AGTCGGTAAC GATTTCGTAA GAGGTGTTTC TGGTGGTGAA
  901 CGTAAGCGTG TTTCCTTGGT TGAAGCTCAG GCAATGAATG CCTCCATCTA CTCTTGGGAT
  961 AACGCCACAA GAGGTTTGGA TGCCTCTACT GCTTTAGAGT TTGCCCAAGC CATTAGAACG
1021 GCTACAAATA TGGTAAACAA CTCTGCTATT GTTGCTATTT ACCAAGCTGG TGAAAATATT
1081 TATGAATTAT TTGATAAAAC TACTGTTCTA TATAACGGTA GACAGATTTA CTTCGGTCCT
1141 GCTGATAAAG CTGTTGGATA TTTCCAAAGA ATGGGTTGGG TTAAACCAAA CAGAATGACC
1201 TCTGCGGAAT TTTTAACATC CGTCACGGTC GATTTTGAAA ATAGGACATT GGATATTAAA
1261 CCTGGCTATG AAGATAAAGT TCCAAAATCT AGTTCAGAGT TTGAGGAATA CTGGTTGAAC
1321 TCTGAGGATT ATCAGGAACT TTTAAGAACT TATGATGATT ATCAAAGTAG ACACCCTGTT
1381 AATGAAACGA GAGATAGACT GGATGTGGCC AAGAAGCAAA GACTGCAACA AGGCCAAAGA
1441 GAAAATTCTC AATATGTTGT CAATTATTGG ACACAAGTTT ATTATTGTAT GATTCGTGGT
1501 TTTCAAAGGG TTAAGGGTGA TTCAACGTAT ACTAAGGTCT ACTTAAGTTC TTTTTTGATC
1561 AAAGCTTTGA TTATCGGTTC TATGTTCCAC AAAATTGATG ACAAAAGTCA ATCCACCACG
1621 GCAGGTGCTT ATTCTCGTGG TGGTATGTTA TTCTATGTTT TATTGTTCGC TTCTGTTACT
1681 TCCTTGGCCG AAATTGGTAA CTCTTTTTCT AGTAGACCTG TTATTGTCAA ACACAAATCA
1741 TATTCCATGT ACCATTIGTC TGCGGAATCG TTACAAGAGA TTATCACTGA GTTCCCTACT
1801 AAATTTGTCG CTATTGTGAT ACTATGTTTG ATTACTTACT GGATTCCATT TATGAAATAT
1861 GAAGCTGGTG CTTTCTTCCA GTATATTTTA TATCTACTGA CTGTGCAACA ATGTACTTCT
1921 TTCATTTTCA AGTTTGTTGC TACTATGAGT AAATCTGGTG TGGATGCCCA TGCCGTCGGT
1981 GGTTTATGGG TCCTGATGCT TTGTGTTTAT GCTGGTTTTG TCTTGCCAAT TGGTGAAATG
2041 CATCATTGGA TTAGATGGCT TCATTTCATT AATCCTTTAA CTTATGCTTT TGAAAGTTTA
2101 GTTTCCACTG AATTTCACCA CAGGGAAATG TTGTGTAGCG CCTTAGTCCC ATCTGGTCCT
2161 GGTTATGAAG GTATTTCTAT TGCTAACCAA GTCTGTGATG CTGCTGGTGC GGTTAAGGGT
2221 AACTTGTATG TTAGCGGTGA CTCTTACATC TTACACCAAT ATCATTTCGC ATATAAGCAT
2281 GCTTGGAGAA ATTGGGGTGT GAACATTGTG TGGACTTTTG GTTATATTGT GTTCAATGTC
2341 ATCTTATCAG AATATTTGAA ACCTGTTGAG GGAGGAGGTG ACTTGCTGTT ATATAAGAGA
2401 GGTCATATGC CGGAGTTAGG TACCGAAAAT GCAGATGCAA GAACCGCTTC CAGAGAGGAA
2461 ATGATGGAGG CTCTGAATGG TCCAAATGTC GATTTAGAAA AGGTCATTGC AGAAAAGGAC
2521 GTTTTCACCT GGAACCATCT GGACTACACC ATTCCATACG ACGGAGCTAC AAGAAAATTA
2581 TTATCGGATG TCTTTGGTTA CGTTAAGCCT GGTAAGATGA CCGCCTTGAT GGGTGAATCC
2641 GGTGCTGGTA AAACTACCTT GTTAAATGTT TTAGCACAAA GAATCAATAT GGGTGTCATC
2701 ACTGGTGATA TGTTAGTCAA TGCCAAGCCC TTGCCTGCTT CTTTCAACAG ATCATGTGGT
2761 TATGTTGCGC AAGCCGATAA TCATATGGCC GAATTATCTG TTAGGGAATC CCTGAGATTT
2821 GCAGCCGAGT TAAGACAGCA AAGTTCCGTT CCGTTAGAGG AGAAATATGA ATATGTTGAA
2881 AAAATTATCA CATTGCTAGG TATGCAAAAT TACGCTGAAG CCTTAGTTGG TAAGACTGGT
2941 AGAGGTTTGA ACGTTGAACA GAGAAAGAAG TTATCTATTG GTGTTGAACT GGTTGCTAAA
3001 CCATCATTAT TATTGTTTTT GGATGAGCCT ACCTCTGGTC TGGACTCTCA GTCTGCTTGG
3061 TCAATTGTTC AATTCATGAG AGCCTTAGCT GATTCTGGTC AATCCATTTT GTGTACGATT
3121 CATCAACCCT CTGCTACCTT GTTTGAACAG TTTGACAGAT TGTTGTTGTT AAAGAAAGGT
3181 GGTAAGATGG TTTACTTTGG TGACATTGGT CCAAATTCTG AAACTTTGTT GAAGTATTTT
3241 GAACGTCAAT CTGGTATGAA GTGTGGTGTT TCTGAAAATC CAGCTGAATA TATTTTGAAT
3301 TGTATTGGTG CCGGTGCCAC TGCTAGTGTT AACTCTGATT GGCACGACTT ATGGCTTGCT
3361 TCCCCAGAAT GTGCCGCTGC AAGGGCTGAA GTTGAAGAAT TACATCGTAC TTTACCTGGT
3421 AGAGCAGTTA ATGATGATCC TGAGTTAGCT ACAAGATTTG CTGCCAGTTA CATGACTCAA
3481 ATCAAATGTG TTTTACGTAG AACAGCTCTT CAATTTTGGA GATCGCCTGT CTATATCAGG
3541 GCCAAATTCT TTGAATGTGT CGCATGTGCT TTGTTCGTCG GTTTATCATA TGTTGGTGTA
3601 AATCACTCTG TTGGTGGTGC CATTGAGGCC TTTTCGTCTA TTTTCATGCT ATTATTGATT
3661 GCTCTGGCTA TGATCAATCA ACTGCACGTC TTCGCTTATG ATAGTAGGGA ATTATATGAG
3721 GTTAGAGAAG CCGCTTCTAA CACTTTCCAT TGGAGTGTCT TGTTATTATG TCATGCTGCT
3781 GTTGAAAACT TTTGGTCCAC ACTTTGTCAG TTTATGTGTT TCATTTGCTA CTACTGGCCA
3841 GCTCAATTCA GTGGACGTGC ATCTCATGCA GGTTTCTTCT TCTTCTTCTA TGTTTTAATT
3901 TTCCCATTAT ATTTTGTCAC ATATGGTCTA TGGATCCTGT ACATGTCTCC TGATGTTCCC
3961 TCAGCTTCTA TGATTAATTC CAATTTGTTT GCTGCTATGT TACTGTTCTG TGGTATTTTA
4021 CAACCAAGAG AGAAAATGCC TGCCTTCTGG AGAAGATTGA TGTATAATGT ATCACCATTT
4081 ACCTACGTGG TTCAAGCTTT GGTTACACCA TTAGTTCACA ATAAAAAGGT CGTTTGTAAT
4141 CCTCATGAAT ACAACATCAT GGACCCACCA AGCGGAAAAA CTTGTGGTGA GTTTTTATCT
4201 ACCTATATGG ACAATAATAC CGGTTATTTG GTAAATCCAA CTGCCACCGA AAACTGTCAA
4261 TATTGCCCAT ACACTGTTCA AGATCAAGTT GTGGCTAAAT ACAATGTCAA ATGGGATCAC
4321 AGATGGAGAA ACTTTGGTTT CATGTGGGCT TATATTTGCT TCAATATTGC CGCTATGTTG
4381 ATTTGTTACT ATGTTGTAAG AGTTAAGGTG TGGTCTTTGA AGTCTGTTTT GAATTTCAAG
4441 AAATGGTTTA ATGGGCCAAG AAAGGAAAGA CATGAAAAAG ATACCAACAT TTTCCAAACA
4501 GTTCCAGGTG ACGAAAATAA AATCACGAAG AAATAA
YOR1 (sequence from strain S288C):
(SEQ ID NO: 3)
      1 ATGACGATTA CCGTGGGGGA TGCAGTTTCG GAGACGGAGC TGGAAAACAA AAGTCAAAAC
    61 GTGGTACTAT CTCCCAAGGC ATCTGCTTCT TCAGACATAA GCACAGATGT TGATAAGGAC
  121 ACATCGTCTT CTTGGGATGA CAAATCTTTG CTGCCTACAG GTGAATATAT TGTGGACAGA
  181 AATAAGCCCC AAACCTACTT GAATAGCGAT GATATCGAAA AAGTGACAGA ATCTGATATT
  241 TTCCCTCAGA AACGTCTGTT TTCATTCTTG CACTCTAAGA AAATTCCAGA AGTACCACAA
  301 ACCGATGACG AGAGGAAGAT ATATCCTCTG TTCCATACAA ATATTATCTC TAACATGTTT
  361 TTTTGGTGGG TTCTACCCAT CCTGCGAGTT GGTTATAAGA GAACGATACA GCCGAACGAT
  421 CTCTTCAAAA TGGATCCGAG GATGTCTATA GAGACCCTTT ATGACGACTT TGAAAAAAAC
  481 ATGATTTACT ATTTTGAGAA GACGAGGAAA AAATACCGTA AAAGACATCC AGAAGCGACA
  541 GAAGAAGAGG TTATGGAAAA TGCCAAACTA CCTAAACATA CAGTTCTGAG AGCTTTATTA
  601 TTCACTTTTA AGAAACAGTA CTTCATGTCG ATAGTGTTTG CAATTCTCGC TAATTGTACA
  661 TCCGGTTTTA ACCCCATGAT TACCAAGAGG CTAATTGAGT TTGTCGAAGA AAAGGCTATT
  721 TTTCATAGCA TGCATGTTAA CAAAGGTATT GGTTACGCTA TTGGTGCATG TTTGATGATG
  781 TTCGTTAACG GGTTGACGTT CAACCATTTC TTTCATACAT CCCAACTGAC TGGTGTGCAA
  841 GCTAAGTCTA TTCTTACTAA AGCTGCCATG AAGAAAATGT TTAATGCATC TAATTATGCG
  901 AGACATTGTT TTCCTAACGG TAAAGTGACT TCTTTTGTAA CAACAGATCT CGCTAGAATT
  961 GAATTTGCCT TATCTTTTCA GCCGTTTTTG GCTGGGTTCC CTGCAATTTT GGCTATTTGC
1021 ATTGTTTTAT TGATCGTTAA CCTTGGACCC ATTGCCTTAG TTGGGATTGG TATTTTTTTC
1081 GGTGGGTTTT TCATATCCTT ATTTGCATTT AAGTTAATTC TGGGCTTTAG AATTGCTGCG
1141 AACATCTTCA CTGATGCTAG AGTTACCATG ATGAGAGAAG TGCTGAATAA TATAAAAATG
1201 ATTAAATATT ATACGTGGGA GGATGCGTAT GAAAAAAATA TTCAAGATAT TAGGACCAAA
1261 GAGATTTCTA AAGTTAGAAA AATGCAACTA TCAAGAAATT TCTTGATTGC TATGGCCATG
1321 TCTTTGCCTA GTATTGCTTC ATTGGTCACT TTCCTTGCAA TGTACAAAGT TAATAAAGGA
1381 GGCAGGCAAC CTGGTAATAT TTTTGCCTCT TTATCTTTAT TTCAGGTCTT GAGTTTGCAA
1441 ATGTTTTTCT TACCTATTGC TATTGGTACT GGAATTGACA TGATCATTGG ATTGGGCCGT
1501 TTGCAAAGCT TATTGGAGGC TCCAGAAGAT GATCCAAATC AGATGATTGA AATGAAGCCT
1561 TCTCCTGGCT TTGATCCAAA ATTGGCTTTA AAAATGACAC ATTGCTCATT TGAGTGGGAA
1621 GATTATGAAT TAAACGACGC TATTGAAGAA GCAAAAGGAG AAGCTAAAGA TGAAGGTAAA
1681 AAGAACAAAA AAAAGCGTAA GGATACATGG GGTAAGCCAT CTGCAAGTAC TAATAAGGCG
1741 AAAAGATTGG ACAATATGTT GAAAGACAGA GACGGCCCGG AAGATTTAGA AAAAACTTCG
1801 TTTAGGGGTT TCAAGGACTT GAACTTCGAT ATTAAAAAGG GCGAATTTAT TATGATTACG
1861 GGACCTATTG GTACTGGTAA ATCTTCATTA TTGAATGCGA TGGCAGGATC AATGAGAAAA
1921 ACTGATGGTA AGGTTGAAGT CAACGGGGAC TTATTAATGT GTGGTTATCC ATGGATTCAA
1981 AATGCATCTG TAAGAGATAA CATCATATTC GGTTCACCAT TCAATAAAGA AAAGTATGAT
2041 GAAGTAGTTC GTGTTTGCTC TTTGAAAGCT GATCTGGATA TTTTACCGGC AGGCGATATG
2101 ACCGAAATTG GGGAACGTGG TATTACTTTA TCTGGTGGTC AAAAGGCACG TATCAATTTA
2161 GCCAGGTCTG TTTATAAGAA GAAGGATATT TATCTATTCG ACGATGTCCT AAGTGCTGTC
2221 GATTCTCGTG TTGGTAAACA CATCATGGAT GAATGTCTAA CCGGAATGCT TGCTAATAAA
2281 ACCAGAATTT TAGCAACGCA TCAATTGTCA CTGATTGAGA GAGCTTCTAG AGTCATCGTT
2341 TTAGGTACTG ATGGCCAAGT CGATATTGGT ACTGTTGATG AGCTAAAAGC TCGTAATCAA
2401 ACTTTGATAA ATCTTTTACA ATTCTCTTCT CAAAATTCGG AGAAAGAGGA TGAAGAACAG
2461 GAAGCGGTTG TTGCCGGTGA ATTGGGACAA CTAAAATATG AATCAGAGGT AAAGGAATTG
2521 ACTGAACTGA AGAAAAAGGC TACAGAAATG TCACAAACTG CAAATAGTGG TAAAATTGTA
2581 GCGGATGGTC ATACTAGTAG TAAAGAAGAA AGAGCAGTCA ATAGTATCAG TCTGAAAATA
2641 TACCGTGAAT ACATTAAAGC TGCAGTAGGT AAGTGGGGTT TTATCGCACT ACCGTTGTAT
2701 GCAATTTTAG TCGTTGGAAC CACATTCTGC TCACTTTTTT CTTCCGTTTG GTTATCTTAC
2761 TGGACTGAGA ATAAATTCAA AAACAGACCA CCCAGTTTTT ATATGGGTCT TTACTCCTTC
2821 TTTGTGTTTG CTGCTTTCAT ATTCATGAAT GGCCAGTTCA CCATACTTTG CGCAATGGGT
2881 ATTATGGCAT CGAAATGGTT AAATTTGAGG GCTGTGAAAA GAATTTTACA CACTCCAATG
2941 TCATACATAG ATACCACACC TTTGGGACGT ATTCTGAACA GATTCACAAA AGATACAGAT
3001 AGCTTAGATA ATGAGTTAAC CGAAAGTTTA CGGTTGATGA CATCTCAATT TGCTAATATT
3061 GTAGGTGTTT GCGTCATGTG TATTGTTTAC TTGCCGTGGT TTGCTATCGC AATTCCGTTT
3121 CTTTTGGTCA TCTTTGTTCT GATTGCTGAT CATTATCAGA GTTCTGGTAG AGAAATTAAA
3181 AGACTTGAAG CTGTGCAACG GTCTTTTGTT TACAATAATT TAAATGAAGT TTTGGGTGGG
3241 ATGGATACAA TCAAAGCATA CCGAAGTCAG GAACGATTTT TGGCGAAATC AGATTTTTTG
3301 ATCAACAAGA TGAATGAGGC GGGATACCTT GTAGTTGTCC TGCAAAGATG GGTAGGTATT
3361 TTCCTTGATA TGGTTGCTAT CGCATTTGCA CTAATTATTA CGTTATTGTG TGTTACGAGA
3421 GCCTTTCCTA TTTCCGCGGC TTCAGTTGGT GTTTTGTTGA CTTATGTATT ACAATTGCCT
3481 GGTCTATTAA ATACCATTTT AAGGGCAATG ACTCAAACAG AGAATGACAT GAATAGTGCC
3541 GAAAGATTGG TAACATATGC AACTGAACTA CCACTAGAGG CATCCTATAG AAAGCCCGAA
3601 ATGACACCTC CAGAGTCATG GCCCTCAATG GGCGAAATAA TTTTTGAAAA TGTTGATTTT
3661 GCCTATAGAC CTGGTTTACC TATAGTTTTA AAAAATCTTA ACTTGAATAT CAAGAGTGGG
3721 GAAAAAATTG GTATCTGTGG TCGTACAGGT GCTGGTAAGT CCACTATTAT GAGTGCCCTT
3781 TACAGGTTGA ATGAATTGAC CGCAGGTAAA ATTTTAATTG ACAATGTTGA TATAAGTCAG
3841 CTGGGACTTT TCGATTTAAG AAGAAAATTA GCCATCATTC CACAAGATCC AGTATTATTT
3901 AGGGGTACGA TTCGCAAGAA CTTAGATCCA TTTAATGAGC GTACAGATGA CGAATTATGG
3961 GATGCATTGG TGAGAGGTGG TGCTATCGCC AAGGATGACT TGCCGGAAGT GAAATTGCAA
4021 AAACCTGATG AAAATGGTAC TCATGGTAAA ATGCATAAGT TCCATTTAGA TCAAGCAGTG
4081 GAAGAAGAGG GCTCCAATTT CTCCTTAGGT GAGAGACAAC TATTAGCATT AACAAGGGCA
4141 TTGGTCCGCC AATCAAAAAT ATTGATTTTG GATGAGGCTA CATCCTCAGT GGACTACGAA
4201 ACGGATGGCA AAATCCAAAC ACGTATTGTT GAGGAATTTG GAGATTGTAC AATTTTGTGT
4261 ATTGCTCACA GACTGAAGAC CATTGTAAAT TATGATCGTA TTCTTGTTTT AGAGAAGGGT
4321 GAAGTCGCAG AATTCGATAC ACCATGGACG TTGTTTAGTC AAGAAGATAG TATTTTCAGA
4381 AGCATGTGTT CTAGATCTGG TATTGTGGAA AATGATTTCG AGAACAGAAG TTAA
CKA2 (sequence from strain S288C):
(SEQ ID NO: 4)
      1 ATGCCATTAC CTCCGTCAAC ATTGAACCAG AAATCTAATA GAGTCTACTC TGTAGCTAGG
    61 GTGTACAAGA ATGCCTGCGA GGAGAGACCA CAAGAATACT GGGACTACGA ACAAGGGGTG
  121 ACCATCGATT GGGGAAAGAT TTCCAATTAC GAAATTATCA ACAAAATTGG AAGAGGGAAA
  181 TATTCCGAAG TGTTCAGCGG TAGATGTATT GTAAACAACC AGAAGTGTGT TATTAAAGTT
  241 TTAAAACCAG TTAAAATGAA AAAAATTTAT AGAGAGTTGA AAATTCTGAC CAATCTAACA
  301 GGCGGCCCCA ATGTTGTTGG CCTTTATGAT ATAGTACAAG ACGCTGACTC CAAAATACCT
  361 GCTTTGATCT TTGAGGAAAT CAAAAATGTT GATTTCAGAA CTTTATATCC TACATTCAAA
  421 CTTCCTGACA TCCAGTATTA TTTCACGCAA TTATTGATTG CGTTAGACTA CTGTCACTCC
  481 ATGGGCATAA TGCACAGAGA CGTAAAGCCT CAGAATGTCA TGATTGATCC TACGGAACGT
  541 AAACTAAGGC TGATCGATTG GGGCCTGGCG GAGTTCTACC ATCCAGGTGT AGATTACAAC
  601 GTTCGTGTCG CTTCGCGTTA CCACAAGGGA CCAGAACTTT TAGTAAACTT GAACCAATAT
  661 GACTACTCCC TAGACTTATG GTCAGTAGGA TGCATGCTAG CAGCTATTGT CTTCAAAAAA
  721 GAACCTTTTT TCAAAGGGTC GTCTAATCCA GATCAACTGG TAAAGATTGC CACAGTACTA
  781 GGAACCAAGG AACTGTTAGG CTATTTGGGT AAGTACGGGT TGCACTTACC ATCTGAATAC
  841 GACAACATTA TGAGAGACTT TACAAAAAAA TCGTGGACAC ACTTTATAAC CTCCGAGACC
  901 AAATTAGCTG TTCCTGAAGT GGTTGATTTA ATCGACAATT TATTAAGGTA TGACCATCAA
  961 GAAAGATTAA CAGCAAAGGA GGCTATGGAT CATAAGTTTT TCAAAACGAA GTTTGAATAA
LCB3 (sequence from strain S288C):
(SEQ ID NO: 5)
      1 ATGGTAGATG GACTGAATAC CTCGAACATT AGGAAAAGAG CCAGGACTCT CTCTAACCCC
    61 AATGACTTTC AAGAGCCTAA TTACTTGCTG GATCCCGGTA ATCATCCCTC AGATCATTTT
  121 CGAACTCGAA TGTCCAAATT TCGGTTTAAT ATTAGAGAGA AGCTGTTAGT GTTTACCAAC
  181 AATCAATCAT TCACATTAAG CCGCTGGCAA AAGAAGTACC GTTCTGCGTT TAATGATCTC
  241 TACTTTACTT ATACTTCCTT AATGGGATCG CATACCTTCT ATGTTCTGTG TTTACCTATG
  301 CCCGTGTGGT TTGGATATTT TGAAACAACA AAAGATATGG TTTATATCTT GGGATATTCT
  361 ATCTACTTGA GTGGTTTTTT TAAAGATTAC TGGTGCTTGC CCAGGCCTAG AGCACCTCCA
  421 TTACATCGAA TTACGTTAAG TGAATATACA ACGAAGGAAT ATGGTGCTCC AAGCTCCCAT
  481 ACAGCAAATG CAACAGGAGT GAGTCTCTTG TTTCTCTACA ACATCTGGAG GATGCAAGAA
  541 TCTTCTGTCA TGGTCCAACT ATTGTTGTCA TGTGTGGTTT TATTTTATTA TATGACTTTG
  601 GTTTTCGGTA GAATATACTG TGGGATGCAT GGCATATTAG ATTTAGTAAG CGGTGGGCTC
  661 ATTGGAATAG TGTGTTTCAT TGTTAGGATG TATTTCAAGT ACAGGTTTCC GGGTTTACGC
  721 ATTGAGGAGC ATTGGTGGTT TCCTTTGTTT AGTGTGGGAT GGGGTCTTCT TCTTTTGTTT
  781 AAACATGTTA AGCCCGTAGA CGAATGTCCT TGCTTCCAAG ATAGTGTTGC GTTCATGGGC
  841 GTTGTGTCAG GTATTGAATG CTGTGATTGG TTGGGCAAAG TGTTTGGAGT CACCCTGGTG
  901 TACAATTTGG AACCTAACTG TGGCTGGCGG TTAACCTTAG CCAGGCTGCT GGTGGGCCTA
  961 CCGTGCGTTG TTATCTGGAA GTACGTGATC AGCAAACCGA TGATCTACAC GTTATTGATC
1021 AAAGTTTTCC ATCTGAAGGA TGACAGAAAC GTTGCGGCAA GAAAAAGACT GGAGGCCACG
1081 CACAAAGAAG GTGCAAGCAA GTACGAATGT CCATTATATA TTGGAGAGCC CAAGATTGAC
1141 ATTCTAGGTA GATTTATTAT CTATGCTGGC GTTCCATTCA CCGTTGTAAT GTGCAGCCCC
1201 GTCCTATTTT CCCTCTTAAA TATAGCATAA
VPS51 (sequence from strain S288C):
(SEQ ID NO: 6)
      1 ATGGCAGAAC AAATTAGTCA CAAAAAGTCC CTGAGGGTGA GCAGTCTGAA CAAAGATAGA
    61 AGATTGCTCT TAAGAGAATT CTACAACTTG GAAAATGAAC CGAATAAAGG TCGTCAAGAA
  121 GCACGTATAG GGGAAAAAGC CAGCGAGGCT CATTCTGGGG AGGAGCAAGT CACAGATGTG
  181 AATATAGATA CCGAAGCAAA CACTGAGAAA CCAGTGAAAG ATGACGAATT GAGTGCAACT
  241 GAAGAGGATC TCAAGGAGGG GTCAGAAGAT GCAGAAGAAG AGATAAAGAA CCTGCCCTTT
  301 AAGAGGCTAG TACAAATTCA CAATAAGCTC TTGGGCAAAG AAACCGAGAC TAACAATTCT
  361 ATCAAGAATA CCATTTACGA AAACTATTAT GACCTAATAA AAGTCAACGA CCTTCTCAAA
  421 GAAATTACAA ACGCCAATGA AGACCAAATT AACAAGTTGA AGCAAACAGT AGAATCTTTA
  481 ATCAAGGAAC TGTAA
VPS52 (sequence from strain S288C):
(SEQ ID NO: 7)
      1 ATGGATGTTC TCAAAGAGGT GTTGTCACTA GACCAAGATA AATTTGACCA GCTGAAGGAA
    61 ACGAGCCGAG ATAAAACAAA TGAAACGGAT GATCCTTTTG AAAACTATTT GAAGGATTGT
  121 AAATTTAAAG CGCCTTCAAA CAAAGATCAG TCACCATTTG CTAAACTTAA ATCATTACAG
  181 GAAACTCATT CTAACAATGA AGCGGCTATT AATATAATTA TTCCTCAATT GATTGATTAC
  241 TTAACCGAAT TCACTAATAG GTTATCAAAT TACACACAAG ATTTAGACTT CATTAAAAAA
  301 AAGTCCAATG AATTACAGTC ATTGCTCGAA TACAACTCCA CTAAACTGGC ACATATCTCT
  361 CCTATGGTTA ATGATTTGAT GATTCCTCCT GAACTCATTG ATGACATCAT TAAAGGGAAG
  421 ATCAATGAAA GCTGGCAGGA TAATATAACA TTCATAGCAG ATAAAGAAGA AATTTATAAC
  481 AAGTATAGGT CCAATAATCT CGATCAAGAC AACAAGGACG CAGAAAATTC AGCAATGCTA
  541 GCACCAAAGG ATTTTGATAA GTTATGTCAA CTCCTGGACA TCCTAAAAAA TGTTATTCTA
  601 GAAAGGTCGA AAAGACTTAT TATTTCCAAA ATCAAAACTT TGAGGAGTCA TAACCCAGTA
  661 CCATCGCAAA GGATACAAAA CAAATTATTA AAAGTTCAAA AAATTTTCCC CTTCATAAGA
  721 GATAATAATC TCTCCTTAGC CCTTGAGTTA AGACAGGCAT ATTGTTACAC AATGAAATGG
  781 TATTATAGAG AATACTTTTC TAGATATATC AGGTCATTGA CTATTTTGCA ATTTCAACAA
  841 ATCGACTCGC AATTTGCATT GGGTAATGGC CTTTCTACAA CTTCAGTGAG TGGGTTTAAC
  901 AATTCACCAT CATTGTTTTT CTCAAATTAT CTAACTACAT CCGCTTCAAA TGCTTTCTAT
  961 AATAAACTCC CTGTAACAGA TGAGAAAATT GATAAATACT TTCAGATAAA GAAAAGATTG
1021 AACATTTTAA CACAAGAAGA CAATACGGTA ATGGTATCCC AAATTGCAGA AAATAACACA
1081 ACGAAAAACT ACATTGAAAT TGGATTTAAA AATTTAAACC TTGCAATTTT AGATAACTGT
1141 ACGGTGGAGT ACCATTTTTT AAAAGATTTC TTCGCTATGA ATGGCGATAA TTTTGAGGAA
1201 ATTAATGGTT TATTGGAACA AATATTTCAA CCAACTTTTG ATGAAGCCAC AACATACACT
1261 CAACAACTGA TCCAATATAA TTATGACATT TTTGGTGTAT TAATAAGTAT TCGTGTGGCC
1321 AATCAATTAC AATTTGAATC AGAAAGGAGA GGAATACCGT CTATGTTTGA TAGTTTCTTG
1381 AATGGTCAAT TAATTCAATT ATGGCCTCGA TTTCAGCAAT TGGTCGATTT TCAATGCGAG
1441 AGCTTACGAA AAGCGGCAAT AACTACAAAT GTGGCAAAAT ATGCCGGCAA CTCAAGCACA
1501 TCCAATAGTA GCCCTTTGAC CTCACCTCAT GAGTTAACTG TACAGTTCGG TAAATTTTTA
1561 TCAAGCTTCT TGACGTTGGC AATAACACAT AAGCAGTCCA TAGACGAAAG ATCTGAACCC
1621 TTATACAATT CCATCATTAG ATTAAGAAAT GATTTCGAAA CAGTCATGAC AAAGTGCAGT
1681 AAAAAGACGA AATCACCAGA AAGATTTCTG GCTACAAATT ACATGTATTT ATACAATAAC
1741 CTACAGCAAT TGCATCTACA TTTAAATATA AATGACTCGG ATGCACAAAA CTACAATTTT
1801 GATTCTGCTG AAAATGTTGG TACGAAAGTT GCGAATGACG ACGATAATGA TTCAAGCGTA
1861 CCACTAATAA TCAGAGAGAC CGAAAATCAT TTCAAAACTT TAGTTGAAGC TTTCACCAGA
1921 AATTGA
VPS53 (sequence from strain S288C)
(SEQ ID NO: 8)
      1 ATGCTGGAAG GTACGGTAGA TTATGACCCG CTGGAAGATA TTACCAATAT ACTTTTTTCA
    61 AAAGAATCCC TGAACAACAT AGATGAACTG ATCAGTATTA CCAGAAGCTA CAAAAAGCAA
  121 TTGCAAGAGG ATATTCTCAA AGAAGAGAAT GAATTGAAGG AACACCCTAA AAATTCCGCT
  181 GAAATAGAGG CTTCTCTGAG GAAAGTTTTC CAAGATTTCA AAGAAACTCA AGATGTCTCA
  241 GCCTCCACCG AGTTGACGAT ATCGAATCTG ACAGAAGGTA TCTCGTACCT GGACATTGCC
  301 AAGAAAAACC TCACCCACTC TTTGACTCTT TTCCAAAATT TAAAGATATT GACAGACAGT
  361 TACATACAAT GCAATGAATT ACTCTCACAG GGCTCATTCA AAAAAATGGT GTCCCCTTAT
  421 AAGATAATGT GTTCGCTTGC TGAAAACACA TTCATCTCTT ACAAATCATT GGACGAGATA
  481 AACTATTTGT TGAGCTCCAT TTCAAGACTG AAAGGAGACA CTTTGTCCAA AATTAAACAA
  541 AACTACAATG CGCTCTTTTC CGGCGGCAAT ATCTCAGAGC ATGATACAGC ACTCACTATG
  601 GAATTGCGCG AAGGTGCCTG CGAGCTACTC GACTGCGATA CAAGTACGAG AGCCCAGATG
  661 ATAGATTGGT GTTTGGACAA ACTTCTCTTC GAAATGAAAG AGATATTTAG GGTCGACGAT
  721 GAAGCCGGAT CCCTAGAAAA TTTATCGAGA AGATACATTT ACTTCAAAAA AATTCTTAAT
  781 AACTTCAATT CAAAGTTCGC AGACTATTTC TTAAAAGACT GGGAAATGGC AGTCAGATTG
  841 ACCACCACTT TTTATCACAT TACACACAAG GACCTTCAGA CACTTCTGAA AAGGGAATTC
  901 AAAGACAAGA ACCCTTCCAT TGATCTATTC ATGACAGCAT TACAATCGAC GCTAGATTTC
  961 GAAAAATACA TCGACGTACG ATTTTCAAAA AAAATTAAGG AACCAAAACT AAGTTCCTGC
1021 TTCGAACCTT ATTTGACTTT ATGGGTGTCT CACCAAAACC AAATGATGGA AAAGAAATTT
1081 CTTTCTTATA TGAGTGAGCC GAAGTACCCA TCTAATGAAA CAGAATCTCT CGTGTTACCC
1141 TCGAGTGCAG ACCTTTTCAG GACATATCGT TCCGTACTGA CTCAGACCTT AGAGCTCATT
1201 GATAATAATG CCAATGATAG CATATTGACT TCATTGGCAA ATTTTTTCAG TAGATGGCTT
1261 CAAACTTACT CACAAAAAAT TCTTCTTCCT TTACTGCTGC CCGACAATAT TGAAGTCCAG
1321 GATAAGCTAG AAGCTGCCAA GTATACCGTT TTATTGATCA ATACTGCAGA TTATTGTGCC
1381 ACGACTATAG ATCAATTGGA GGATAAATTA TCTGAATTCA GCGGTAATCG TGAAAAGCTG
1441 GCAAACAGTT TTACGAAAAC GAAAAATATA TACGACGATT TACTAGCAAA AGGAACTTCT
1501 TTTCTATTAA ACCGTGTCAT ACCCTTAGAT CTAAATTTTG TATGGAGAGA GTTTATCAAC
1561 AATGATTGGT CAAATGCTGC GATAGAAGAT TATAGCAGGT ACATGGTAAC CCTCAAATCC
1621 GTACTTAAAA TGCCCGCATT AACAGATGCC TCTATTAAAC AACAGCAAGA GCAACCTTCG
1681 ACTTTGGCAT TTATTTTGTC GCAATTCAAT AGAGATGTTT ATAAGTGGAA TTTCTTGGAT
1741 AAGGTGATTG ATATCATCAC TACAAATTTT GTAAGCAATA CCATCCGCCT TCTGCAGCCC
1801 GTTCCACCCT TTTCCCTGGC GGGCAGCAAA AGGAAATTTG AAACCAGAAC TGTTGTCAAC
1861 ATTGGCGAGC AGCTTCTCCT TGATTTAGAA TTGCTGAAGG AGATTTTTCA CACTTTACCA
1921 GAAAGTGTAA GTAACGATTC TGACTTGCGA GAAAATACCT CTTATAAGAG GGTGAAAAGA
1981 CATGCAGACA ATAATATAGA CCAGCTGCTG AAGTTTATTA AACTTCTAAT GGCTCCTCTG
2041 GATTCCGCTG ATGACTATTA CGAGACCTAC TCCAAATTGA CCAATAATAA CCCTGATTCA
2101 GCGGTATGGT CTTTTGTCCT CGCTTTAAAG GGCATTCCAT GGGACCTGGC ATTATGGAAA
2161 AAGCTATGGA GTGCCTACAA CTTAGAAACA GACGACACTG ACGAGGGCAG CAGGCCAGAC
2221 AGTAATCGCG ATCTTTTCAT ATTCAAGTGG GACAAGGTAC TTTTGGGTCA ATTTGAAAAC
2281 AACTTGGCAA GGATGCAAGA TCCGAATTGG TCAAAATTTG TGAGGCAAGA TCTGAAAATA
2341 TCACCACCTG TTATGAAGAG GATAGTATCC ACCCCTCAAA TACAACAACA AAAAGAAGAA
2401 CAAAAAAAGC AAAGTTTGAG TGTCAAAGAC TTCGTTTCTC ACTCAAGGTT CTTTAACAGA
2461 GGCACTTGA
```

The selectable biosynthetic marker genes (auxotrophy needs and/or resistances) can be introduced into the loci of the wild type genes by recombinant DNA techniques. Suitable selectable markers are the below mentioned auxotrophy and resistance markers. Such modified alleles can then be transformed into Saccharomyces cerevisiae, where they replace the wild type loci by homologous recombination. The strains comprising modified alleles can be established by selecting for the biosynthetic marker or markers.

In a specific embodiment, gene deletions in yeast, specifically deletion due to multiple gene knock-outs may be performed using loxP marker cassettes carrying the genes URA3 and LEU2 from Kluyveromyces lactis, his5⁺ from Schizosaccharomyces pombe and the dominant resistance marker ble^{r} from the bacterial transposon Tn5 conferring resistance to the antibiotic pleomycin as described in Gueldener U, 2002. Specifically, the target genes are CKA2, LCB3, VPS51, VPS52, or VPS53.

Furthermore, the target genes each may be a DNA sequence which is at least 90%, specifically at least 95%, specifically 99%, 99.5% or 99.9% identical to the sequences listed above.

Said sequence is able to hybridize under stringent conditions with a probe that can be prepared from any of the aforementioned nucleotide sequences, such as a sequence complementary to the whole sequence or a partial sequence of any of the aforementioned nucleotide sequences, so long as the original function is maintained. The “stringent conditions” refer to conditions under which a so-called specific hybrid is formed, and a non-specific hybrid is not formed. Examples of the stringent conditions include those under which highly homologous DNAs hybridize to each other, for example, DNAs not less than 80% homologous, preferably not less than 90% homologous, more preferably not less than 95% homologous, still more preferably not less than 97% homologous, particularly preferably not less than 99% homologous, hybridize to each other, and DNAs less homologous than the above do not hybridize to each other, or conditions of washing of typical Southern hybridization, i.e., conditions of washing once, preferably 2 or 3 times, at a salt concentration and temperature corresponding to 1×SSC, 0.1% SDS at 60° C., preferably 0.1×SSC, 0.1% SDS at 60° C., more preferably 0.1×SSC, 0.1% SDS at 68° C.

The probe used for the aforementioned hybridization may be a part of a sequence that is complementary to any one of the genes as described above. Such a probe can be prepared by PCR using oligonucleotides prepared on the basis of a known gene sequence as primers and a DNA fragment containing the nucleotide sequence as a template. As the probe, for example, a DNA fragment having a length of about 300 bp can be used. When a DNA fragment having a length of about 300 bp is used as the probe, in particular, the washing conditions of the hybridization may be, for example, 50° C., 2×SSC and 0.1% SDS.

Further, the target genes each may be a gene having any of the aforementioned nucleotide sequences in which an arbitrary codon is replaced with an equivalent codon. For example, the target genes each may be a gene modified so that it has optimal codons according to codon frequencies in a host to be used.

The percentage of the sequence identity between two sequences can also be determined by, for example, using a mathematical algorithm. Non-limiting examples of such a mathematical algorithm include the algorithm of Myers and Miller (1988), a modified version of the algorithm of Karlin S and Altschul S F (1990), such as that described in Karlin S and Altschul S F (1993).

By using a program based on such a mathematical algorithm, sequence comparison (i.e. alignment) for determining the sequence identity can be performed. The program can be appropriately executed by a computer. Examples of such a program are well known to the skilled person, including, but not limited to, CLUSTAL of PC/Gene program, ALIGN program (Version 2.0), and BLAST, FASTA, and TFASTA.

In order to obtain a nucleotide sequence homologous to a target nucleotide sequence, in particular, for example, BLAST nucleotide search can be performed by using BLASTN program with score of 100 and word length of 12. In order to obtain an amino acid sequence homologous to a target protein, in particular, for example, BLAST protein search can be performed by using BLASTX program with score of 50 and word length of 3. See ncbi.nlm.nih.gov for BLAST nucleotide search and BLAST protein search. In addition, Gapped BLAST can be used in order to obtain an alignment including gap(s) for the purpose of comparison. In addition, PSI-BLAST can be used in order to perform repetitive search for detecting distant relationships between sequences. When using BLAST, Gapped BLAST, or PSI-BLAST (Position-Specific Iterative Basic Local Alignment Search Tool), initial parameters of each program (e.g. BLASTN for nucleotide sequences, and BLASTX for amino acid sequences) can be used. Alignment can also be manually performed.

The sequence identity between two sequences is calculated as the ratio of residues matching in the two sequences when aligning the two sequences so as to fit maximally with each other.

According to a specific embodiment, the genes can be disrupted or deleted using CRISPR/Cas9, specifically a multiplex CRISP/Cas9 system, thereby allowing a targeted- and marker-free genome engineering resulting in the fumonisin-sensitive strain as described herein. Said methods are known by the skilled person (Jakociunas T et al., 2015).

In a further specific embodiment, gene disruptions can be performed by inserting selection marker genes such as but not limited to LEU2, URA3, LYS2, ADE2, TRP1, and HIS3, into target genes, specifically into the SNQ2, PDR12 and YOR1 genes.

The URA3 gene encodes orotidine-5′-phosphate decarboxylase, an essential enzyme in pyrimidine biosynthesis in Saccharomyces cerevisiae, the HIS3, LEU2, TRP1, and MET15 marker genes encode essential enzymes for de novo synthesis of the amino acids L-histidine, L-leucine, L-tryptophan, and L-methionine, respectively.

Alternatively, also marker genes can be used which confer resistance against antibiotics or other toxic compounds, e.g. fluoroacetate, hygromycin, sulfometuron, zeocin, kanamycin, or hygromycin or genes which cause resistance, for example, against G418 (aminoglycoside phosphotransferase gene). Selection for strains that carry such marker genes requires the addition of these toxic compounds to the growth media.

The yeast of the invention is not particularly limited as long as it can be modified according to the invention and can be used for the methods of the present invention. The yeast may be budding yeast, or may be fission yeast. The yeast may be haploid yeast, or may be diploid or polyploid yeast. Specifically, the yeast is Saccharomyces cerevisiae, Schizosaccharomyces pombe, or Ustilago maydis.

As used herein, the term “haploid” refers to haploid yeast cells having one copy of each chromosome, i.e. a single set of unpaired chromosomes.

As used herein, the term “diploid” refers to diploid yeast cells having two homologous copies of each chromosome. In a diploid state the haploid number is doubled, thus, this condition is also known as 2n.

“Homologous chromosomes” or “homologous copies of each chromosome” means that the chromosomes have the same genes in the same loci where they provide points along each chromosome which enable a pair of chromosomes to align correctly with each other. However, the chromosomes (and genes) are not necessarily identical. The same gene can be coded by two different alleles. An allele is the variant form of a given gene.

Unlimited examples of Saccharomyces strains, which can be used to produce the yeast mutants described herein and to express heterologous genes, include YPH499, YPH500, YPH501, YNN216, W303, many of them derived from S288C (Louis E, 2016)

Fumonisins are mycotoxins produced by a number of Fusarium sp. or Aspergillus sp. The fumonisin scaffold is comprised of a C18 polyketide backbone functionalized with two tricarballylic esters and an alanine derived amine. These functional groups contribute to fumonisin's ability to inhibit sphingolipid biosynthesis in animals, plants and yeasts. Less toxic fumonisins with a modified amino group compared to FB₁. Toxicity may be diminished by blocking the amine, e.g. by N-acetylation in A-type fumonisins or by formation of a hydroxypyridine structure in P-type compounds. The hydroxyl groups at R1 and R2 are introduced sequentially, in the typically formed mixtures the amounts of FB₁>FB₂>FB₃>FB₄ (lacking both hydroxyl groups). The fumD esterase detoxifies FB₁ by hydrolysis of the tricarballylic acid side chains. The Aspergillus amine oxidase detoxifies by conversion of the NH2 group into a keto group.

The structure of fumonisin B is as follows:

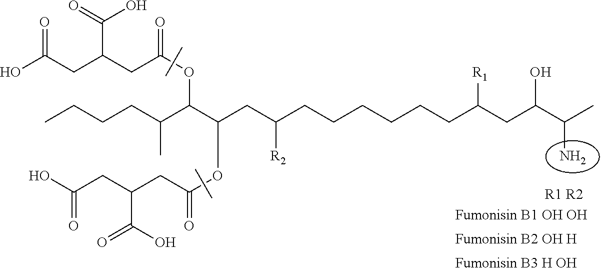

The recombinant fumonisin-sensitive yeast of the present invention displays increased fumonisin sensitivity, specifically it is sensitive against mycotoxins fumonisin B₁, B₂, B₃, B₄, B₅ and FCs.

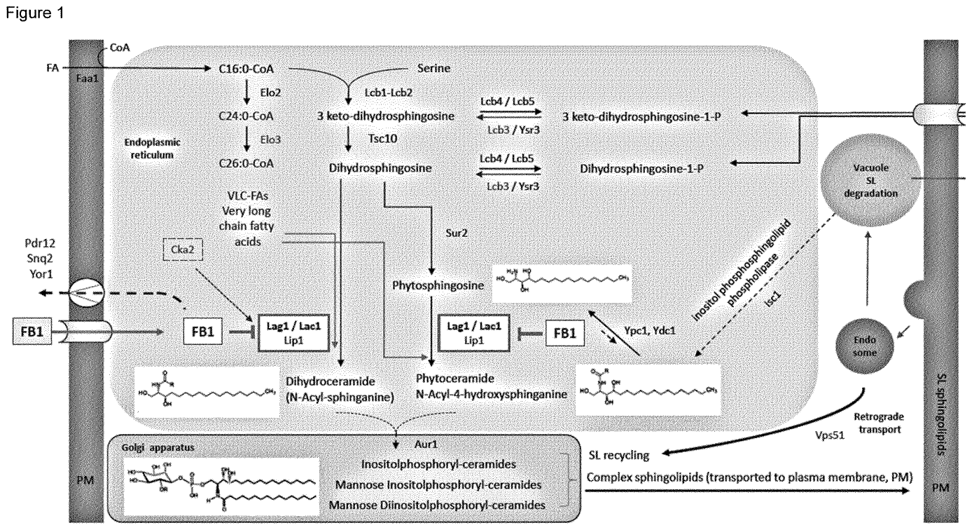

Yeast, specifically baker's yeast Saccharomyces cerevisiae, specifically used herein, serves as an ideal indicator organism for detecting fumonisins, being also highly suited as host for heterologous gene expression. High throughput tools for growth monitoring yeast growth on agar or in liquid culture in microtiter plates, based on turbidity OD600, are available. The sphingolipid biosynthesis pathway in baker's yeast and plants differs from that in animals, since the conversion of dihydroceramide to ceramide by dihydroceramide desaturase occurs only in mammalian cells (Rego A et al, 2013) and also the formation of complex head groups is different. The sphingolipid biosynthesis pathway in yeast is shown in FIG. 1: The wild-type genes coding for sphingolipid biosynthetic enzymes are shown in uppercase italics. Introduced mutations (loss of function) causing fumonisin-sensitivity are shown in bold and underlined in lower case letters with a prefixed delta sign. Hydrophilic fumonisin B₁ (FB₁) can enter yeast cells in unknown ways (possibly utilizing anion/tricarboxylic acid transporters). Intracellular FB₁ in yeast is effluxed back across the plasma membrane by ABC transporter proteins. Triple mutants pdr12 snq2 yor1 show only slightly increased FB₁ sensitivity. The target of FB₁ (possibly a competitive inhibitor) is ceramide synthase. Two catalytic subunits, encoded by LAG1 and LAC1 with differences in substrate preference regarding very long chain fatty acids (VLC-FAs) interact with a common subunit, encoded by LIP1, which is required for enzymatic activity. Casein kinase 2 phosphorylates and thereby activates ceramide synthase. Loss of function mutants of the alpha′ subunit (cka2) show increased FB₁ sensitivity. Ceramide synthase catalyzes the transfer of very-long-chain fatty acyl-CoA to sphingoid bases. The initial step for synthesis of the long chain bases is catalysed by serine palmitoyltransferase (consisting of the two subunits encoded by LCB1 and LCB2). TSC10 encodes 3-ketosphinganine reductase which is essential for growth in the absence of exogenous dihydrosphingosine or phytosphingosine. SUR2 encodes sphinganine C4-hydroxylase. Phosphorylated forms and precursors of the long chain bases can be taken up to a limited extent from the medium. Inactivation of one of the phosphatases needed to dephosphorylate externally supplied phospho-sphingobases leads to lower FB₁ resistance of lcb3 mutants. Also reducing the supply of very long chain bases leads to increased sensitivity to FB₁ in elo3 mutants (not used in the final strain). The product of the essential AUR1 gene (encoding phosphatidylinositol: ceramide phosphoinositol transferase) is needed for synthesis of complex sphingolipids that are preferentially localized in the plasma membrane.

Complex sphingolipids can be recycled from the plasma membrane to the Golgi when retrograde transport is possible. This is blocked in the vps51 mutant, where the endosomes are routed to the vacuole, where degradation takes place. The ISC1 encoded protein (localized in the ER membrane and in mitochondria) is an inositol phosphosphingolipid phospholipase that hydrolyses complex sphingolipids, allowing reuse of the building blocks generated by vacuolar degradation after another round of ceramide synthase. Alkaline dihydroceramidase, encoded by YDC1, preferentially hydrolyzes dihydroceramide to a free fatty acid and dihydrosphingosine, the product of its paralog YPC1 has specificity for phytoceramide. These enzymes have minor reverse activity and when overexpressed can increase FB₁ resistance. Long chain fatty acyl-CoA synthetase (encoded by FAA1 and orthologs) can import and activate fatty acids from the medium for synthesis of sphingoid long-chain bases.

The inactivation of the two ceramide synthase genes (LAG1, LAC1) in yeast leads to lethality (double mutants are viable in some genetic backgrounds but are extremely slow growing). Therefore, when FB₁ inhibits Lag1p and Lac1p, growth inhibition is expected.

Yet, wild-type S. cerevisiae is highly resistant to fumonisins, such as fumonsin B₁, possibly also to fumonisin B₂ or B₃, in particular on rich media containing yeast extract, which can most likely supplement ceramides and biosynthetic precursors that mask the sphingolipid biosynthesis deficiency caused by FB₁.

Yeast, specifically S. cerevisiae, has a low capacity to chemically modify and thereby detoxify harmful substances, but is nevertheless highly resistant to many inhibitors due to a drug efflux system, which is mainly mediated by members of different classes of ABC transporter proteins

Fumonisin, specifically FB₁, can enter yeast cells in unknown ways, possibly utilizing anion/tricarboxylic acid transporters. Intracellular cytotoxic drugs in yeast are effluxed back across the plasma membrane by ATP-binding cassette (ABC) transporter proteins. The yeast genome contains about 30 ABC proteins. Of these proteins, 22 are predicted to contain multiple membrane spans and are thus considered to be true ABC transporters, belonging to the family of ABCB (transporter: MDL1, MDL2, ATM1, STE6), ABCC (transporter: VMR1, YBT1, NFT1, YCF1, BPT1, YOR1), ABCD (transporter: PXA1, PXA2), and ABCG (transporter: PDR5, PDR15, PDR10, SNQ2, PDR18, PDR12, PDR11, AUS1, YOL075c, ADP1) (Paumi C M et al, 2009, Bauer B E et al., 1999).

The target of FB₁, possibly a competitive inhibitor, is ceramide synthase. Two catalytic subunits, Lag1 and Lac1 proteins with differences in substrate preference regarding very long chain fatty acids (VLC-FAs) interact with a common subunit, Lip1, needed for enzymatic activity. The CKA2 gene encodes an alpha′ subunit of casein kinase 2.

The term casein kinase 2 refers to a protein having an activity of catalyzing the serine/threonine-selective phosphorylation of proteins (EC 2.7.11.1). This activity may be referred to as “casein kinase 2 activity”. Cka2 protein may form a heterotetramer in combination with CKA1, CKB1, and CKB2 gene products, i.e. Cka1p, Ckb1p, and Ckb2p, to function as casein kinase 2. Cka2p may be required for full activation of ceramide synthase (Kobayashi S D and Nagiec M M, 2003). Casein kinase 2 activity can be measured by, for example, a known method (Matsuura A et al., 1997). Casein kinase 2 phosphorylates and thereby activates ceramide synthase. Ceramide synthase catalyzes the transfer of very-long-chain fatty acyl-CoA to sphingoid bases. The initial step for synthesis of the long chain bases is catalyzed by serine palmitoyltransferase (consisting of the two subunits Lcb1 and Lcb2). TSC10 encodes 3-ketosphinganine reductase which is essential for growth in the absence of exogenous dihydrosphingosine or phytosphingosine. SUR2 encodes sphinganine C4-hydroxylase. Phosphorylated forms and precursors of the long chain bases can be taken up to a limited extent from the medium.

Sphingolipid long-chain bases (LCBs) are also produced by dephosphorylation of sphingolipid long-chain base phosphates LCBPs in yeast having phyto-Sph1P and dihydro-Sph1P. Two lipid phosphatase families, SPP and LPP, are capable of this reaction, in yeast most of the in vivo LCBP dephosphorylation activity is attributable to SPP proteins. Yeast expresses two SPP family members, Lcb3 and Ysr3, though most of the activity is attributed to Lcb3 (Hirabayashi Y et al., 2006). The LCB3 gene is predicted to encode a protein with multiple membrane-spanning domains and a COOH-terminal glycosylphosphatidylinositol cleavage/attachment site. Deletion of the lcb3 gene in a wild type genetic background reduces the rate of exogenous long chain base incorporation into sphingolipids and makes the host strain more resistant to growth inhibition by long chain bases (Qie L et al, 1997).

Also reducing the supply of very long chain bases leads to increased sensitivity to FB₁ in elo3 mutants, however elo3 mutants showed synthetic growth defects or even synthetic lethality, specifically when combined with other mutations.

The product of the essential AUR1 gene (encoding phosphatidylinositol: ceramide phosphoinositol transferase) is needed for synthesis of complex sphingolipids that are preferentially localized in the plasma membrane. Complex sphingolipids can be recycled to the Golgi when retrograde transport is possible. This is blocked if any one of the VPS51, VPS2, VPS3, and/or VPS54 genes are knocked-out, where the endosomes are routed to the vacuole, where degradation takes place. The Isc1 protein (localized in the ER membrane and in mitochondria) is an inositol phosphosphingolipid phospholipase that hydrolyses complex sphingolipids, allowing reuse of the building blocks generated by vacuolar degradation after another round of ceramide synthase. Alkaline dihydroceramidase, Ydc1 protein, preferentially hydrolyzes dihydroceramide to a free fatty acid and dihydrosphingosine, its paralog Ypc1 has specificity for phytoceramide. These enzymes have minor reverse activity and when overexpressed can increase FB₁ resistance. Long chain fatty acyl-CoA synthetase (Faa1 and orthologs) can import and activate fatty acids from the medium for sphingoid long-chain bases. Inactivation of VPS51, coding for a subunit of the Golgi-associated retrograde protein complex required for retrograde traffic from the early endosome back to the late Golgi; which is involved in vesicle organization and sphingolipid homeostasis by recycling sphingolipids from the plasma membrane also leads to increased susceptibility to fumonisin (Fröhlich F et al., 2015).

Thus, some single mutations in yeast had already been described that inhibited the transport system for expelling toxic substances from the cell. In connection with the mycotoxin fumonisin, however, it has not yet been possible to develop yeasts showing sufficient growth in cell culture and being highly sensitive to fumonisin, thereby being highly useful as bioassay systems for detecting fumonisins in a sample.

According to the invention it was surprisingly shown that
deletion or disruption of ABC transporter proteins YOR1, SNQ2, PDR12 encoding genes, in combination with
disruption or deletion of the CKA2 gene encoding alpha′ catalytic subunit of casein kinase 2, and
disruption or deletion of the LCB3 gene, encoding a long-chain base-1-phosphate phosphatase,
provided a yeast strain which shows highly increased FB₁ sensitivity compared to wild type yeast and yeast having one or two gene disruptions or deletions in any one of the above genes.

Disruption or deletion of any one of VPS51, VPS52, VPS53 or VPS54 genes, coding for a subunit of the Golgi-associated retrograde protein complex required for retrograde traffic from the early endosome back to the late Golgi can further increase fumonisin sensitivity.

The mutant combining these alterations displays FB₁ sensitivity similar to animal cells, yet shows robust growth, having an assay time below 24 h, and can still be used as host for transformation and heterologous gene expression.

Said fumonisin-sensitive yeasts show growth inhibition at fumonisin concentrations of ≥5 μM fumonisin, ≥8 μM fumonisin, ≥10 μM fumonisin, specifically ≥15 μM, ≥20 μM, ≥25 μM, ≥50 μM, ≥100 M, ≥500 μM, ≥1000 μM whereas the wild type yeast does not show significant growth inhibition even at a concentration of 1000 μM FB₁ or more.

Growth rates and growth inhibition can be determined by any method well known in the art, such as, but not limited to, determining cell number, using e.g. a hemocytometer, determining cell viability, e.g. using methylene blue staining, or determining relative growth calculated as the resultant optical density ratio of toxin treated cultures to untreated controls. Also yeast dry weight can be determined or conductance change can be measured during yeast growth, employing direct and indirect methods.

The fumonisin-sensitive yeast described herein can be used for expressing heterologous polypeptides.

The term “expression” is understood in the following way. Nucleic acid molecules containing a desired coding sequence of an expression product such as e.g., a recombinant protein as described herein, and control sequences such as e.g., a promoter in operable linkage, may be used for expression purposes. Hosts transformed or transfected with these sequences are capable of producing the encoded proteins. In order to effect transformation, the expression system may be included in a vector; such as a plasmid, or the relevant DNA is integrated into the host chromosome.

“Expression constructs” or “vectors” or “plasmid” used herein are defined as DNA sequences that are required for the transcription of cloned recombinant nucleotide sequences, i.e. of recombinant genes and the translation of their mRNA in the host organism. Expression vectors or plasmids usually comprise an origin for autonomous replication in the host cells, selectable markers (e.g. an amino acid synthesis gene or a gene conferring resistance to antibiotics), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences.

The term “heterologous” as used herein with respect to a nucleotide or amino acid sequence or protein, refers to a compound which is either foreign, i.e. “exogenous”, such as not found in nature, to the yeast host cell described herein; or that is naturally found in a given host cell, e.g., is “endogenous”, however, in the context of a heterologous construct, e.g. employing a heterologous nucleic acid. The heterologous nucleotide sequence as found endogenously may also be produced in an unnatural, e.g. greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. An example of a heterologous sequence is polynucleotide encoding a protein of interest having detoxifying or resistance conferring properties in the yeast cell. The yeast of the invention also provides a system for screening such proteins of interest.

The term “variant” as used herein in the context of the present invention shall specifically refer to any sequence derived from a parent sequence, e.g. by size variation, e.g. elongation or fragmentation, mutation, hybridization (including combination of sequences), or with a specific degree of homology, or analogy.

The term “native” as used herein in the context of the present invention shall specifically refer to an individual structure or component of an organism, which is naturally associated with its environment. It is, however, well understood, that native structures or components may be isolated from the naturally associated environment, and provided as isolated native structures or components. Such isolated native structures or components may as well be of artificial or synthetic origin, and still have the same characteristics as the ones of natural origin.

The fumonisin-sensitive yeast described herein can be used for expressing heterologous sequences. Because yeast is a well-established host organism, introduction and expression of heterologous genes are well known by the skilled person.

The term “gene” as used herein refers to a DNA sequence that comprises at least promoter DNA, optionally including operator DNA, and coding DNA which encodes a particular amino acid sequence for a particular polypeptide or protein. Promoter DNA is a DNA sequence which initiates, regulates, or otherwise mediates or controls the expression of the coding DNA. Promoter DNA and coding DNA may be from the same gene or from different genes, and may be from the same or different organisms.

The term “recombinant” as used herein shall mean “being prepared by or the result of genetic engineering”. A recombinant host or cell specifically comprises a recombinant expression vector or cloning vector, or it has been genetically engineered to contain a recombinant nucleic acid sequence, in particular employing nucleotide sequence foreign to the host. A recombinant protein is produced by expressing a respective recombinant nucleic acid in a host.

The term “enzyme” in accordance with the invention means any substance composed wholly or largely of protein or polypeptides that catalyzes or promotes, more or less specifically, one or more chemical or biochemical reactions.

Herein provided is also the use of the inventive modified fumonisin-sensitive yeast in a method for detecting inhibitory activity of fumonisins in a sample, comprising the steps of contacting the fumonisin-sensitive yeast with the sample; determining the growth of said yeast in the presence and absence of said sample; wherein reduced growth indicates the presence of fumonisin in the sample.

Any sample, probe or material can be used in this method as long as the concentration of fumonisin is high enough to lead to a reduction in the growth of sensitive yeasts. In particular, this concentration is at least 10 μM, specifically at least 50 μM, specifically at least 100 μM.

As referred herein, “contacting” means that a sample suspected of containing fumonisin is added to the culture medium either as a concentrate or as a diluted preparation. This can be done in a variety of ways, for example by mixing it into, or by adding it to the yeast cultivation medium. The fumonisin-sensitive yeast is cultivated under conditions allowing the yeast cells to proliferate and for a sufficiently long period of time to demonstrate different growth rates in the presence and absence of fumonisin. Specifically, the time period is 12 to 96 hours, specifically 24 to 48 hours, specifically it is more than 48 hours.

Specifically, the sample comprises food extracts, food concentrates, plant extracts, such as, but not limited to corn extracts such as, but not limited to, corn, rice, sorghum and barley, extracts of fungal cultures, stored paddy (Oryza sativa), etc.

Herein provided is also the use of the inventive fumonisin-sensitive yeast in a method for detecting fumonisin detoxifying or resistance conferring compounds in a sample, comprising the steps of contacting the fumonisin-sensitive yeast of the invention with the sample in the presence of fumonisin; determining growth of said yeast in the presence and absence of said sample; wherein increased growth indicates the presence of fumonisin detoxification or resistance conferring compounds in the sample.

Detoxifying refers to the removal of fumonisin by the yeast cellular metabolism, which can be modified due to the expression of a heterologous polypeptide, such as an enzyme or a low molecular compound reacting with fumonisin. Detoxification enzymes are proteins produced from the expression of detoxification genes. Detoxification enzymes act in the cellular metabolism of substances that are strange to the organism (xenobiotic) and endogenous compounds that could cause cellular and tissue damage.

Resistance conferring refers to resistance against fumonisin at concentrations that would otherwise cause decreased cell growth or cell death. Non limiting examples are oxidases, stress response mimetics, etc.

Samples applicable for detecting detoxifying or resistance conferring compounds can be extracts of fungi, crude protein preparations, or purified proteins.

Herein provided is also the use of the inventive fumonisin-sensitive yeast in a method for method for screening the fumonisin detoxification or resistance conferring activity of a heterologous polypeptide, comprising the steps of introducing a heterologous DNA sequence encoding a potential fumonisin detoxification or resistance conferring polypeptide into the fumonisin-sensitive yeast; expressing the potential fumonisin detoxification polypeptide in said yeast; determining the growth of said yeast; whereby growth in the presence of fumonisin of said yeast compared to a reference yeast lacking said heterologous DNA sequence indicates detoxification activity of the polypeptide.

The fumonisin detoxification or resistance conferring polypeptide can be, but is not limited to enzymes, oxidases, such as amine oxidases, N-acetyltransferases, or esterases.

Specifically, the detoxification or resistance conferring activity of one or more polypeptides is screened, wherein the one or more heterologous DNA sequence encoding said one or more polypeptides are introduced into the same fumonisin-sensitive cell or in separate fumonisin-sensitive yeasts such as a library of yeast cells which can be screened for expression of relevant polypeptides.

The medium to be used is not particularly limited, so long as the yeast of the present invention can proliferate in it, and an objective substance can be produced. As the medium, for example, a usual medium used for cultivating yeast can be used. Examples of such a medium include SD medium, SC (synthetic complete) medium, specifically SC medium lacking supplements necessary for selection of auxotrophic marker (SC-LEU, SC-URA. SC-TPR) and YPD medium. The medium may contain carbon source, nitrogen source, phosphate source, and sulfur source, as well as components selected from other various organic components and inorganic components as required. The types and concentrations of the medium components can be appropriately determined according to various conditions such as the type of the yeast to be used and the type of the objective substance to be produced.

Specific examples of the carbon source include, for example, saccharides such as glucose, fructose, sucrose, galactose, arabinose, blackstrap molasses, starch hydrolysates, and hydrolysates of biomass, organic acids such as acetic acid, fumaric acid, citric acid, and succinic acid, alcohols such as glycerol, crude glycerol, and ethanol, and aliphatic acids. As the carbon source, a single kind of carbon source may be used, or two or more kinds of carbon sources may be used in combination.

Specific examples of the nitrogen source include, for example, ammonium salts such as ammonium sulfate, ammonium chloride, and ammonium phosphate, organic nitrogen sources such as peptone, yeast extract, meat extract, and soybean protein decomposition products, ammonia, and urea. Ammonia gas or aqueous ammonia used for adjusting pH may also be used as the nitrogen source. As the nitrogen source, a single kind of nitrogen source may be used, or two or more kinds of nitrogen sources may be used in combination.

Specific examples of the phosphate source include, for example, phosphoric acid salts such as potassium dihydrogen phosphate and dipotassium hydrogen phosphate, and phosphoric acid polymers such as pyrophosphoric acid. As the phosphate source, a single kind of phosphate source may be used, or two or more kinds of phosphate sources may be used in combination.

Specific examples of the sulfur source include, for example, inorganic sulfur compounds such as sulfates, thiosulfates, and sulfites, and sulfur-containing amino acids such as cysteine, cystine, and glutathione. As the sulfur source, a single kind of sulfur source may be used, or two or more kinds of sulfur sources may be used in combination.

Specific examples of other various organic components and inorganic components include, for example, inorganic salts such as sodium chloride and potassium chloride; trace metals such as iron, manganese, magnesium, and calcium; vitamins such as vitamin B1, vitamin B2, vitamin B6, nicotinic acid, nicotinamide, and vitamin B12; amino acids; nucleic acids; and organic components containing those such as peptone, casamino acid, yeast extract, and soybean protein decomposition product. As other various organic components and inorganic components, a single kind of component may be used, or two or more kinds of components may be used in combination.

Further, when an auxotrophic mutant that requires an amino acid, a nucleic acid, or the like for growth thereof is used, it is preferable to supplement a required nutrient to the medium.

The culture conditions are not particularly limited so long as the yeast of the present invention can proliferate, and the objective substance can be produced. The culture can be performed, for example, under usual conditions used for cultivating yeast. The culture conditions can be appropriately determined according to various conditions such as the type of yeast to be used and the type of objective substance to be produced.

The culture can be performed by using microtiter plates under an aerobic condition. Specifically, a preculture with the yeast cells of the invention is diluted such that after addition of the fumonisin (in at least 25% to 50% of the medium), an initial OD of about 0.01 is obtained which is transferred to the cultivation medium. Specifically, the dishes are shaken repeatedly and cell numbers are determined. The term “aerobic condition” may refer to a condition where the dissolved oxygen concentration in the liquid medium is 0.33 ppm or higher, or preferably 1.5 ppm or higher. In cases of the aerobic condition, the oxygen concentration can be controlled to be, for example, 5 to 50%, preferably about 10 to 20%, of the saturated oxygen concentration. Specifically, the aerobic culture can be performed with aeration or shaking.

The culture temperature may be, for example, 25 to 35° C., preferably 27 to 33° C., more preferably 28 to 32° C. pH of the medium may be, for example, 3 to 8 or 4 to 6. pH of the medium may be adjusted as required during the culture. For adjusting pH, inorganic or organic acidic or alkaline substances, such as ammonia gas and so forth, can be used. The culture period may be, for example, 10 to 200 hours, or 15 to 120 hours. The culture condition may be constant during the whole period of the culture, or may be changed during the culture. The culture can be performed as batch culture, fed-batch culture, continuous culture, or a combination of these. Further, the culture may be performed as two steps of a seed culture and a main culture. In such a case, the culture conditions of the seed culture and the main culture may or may not be the same. For example, both the seed culture and the main culture may be performed as batch culture.

### EXAMPLES

The examples described herein are illustrative of the present invention and are not intended to be limitations thereon. Different embodiments of the present invention have been described according to the present invention. Many modifications and variations may be made to the techniques described and illustrated herein without departing from scope of the invention. Accordingly, it should be understood that the examples are illustrative only and are not limiting upon the scope of the invention.

### Example 1: Strain Generation

The strain construction was started in the genetic background of the frequently used yeast laboratory strains YPH499 and YPH500 (Sikorski R S and Hieter P, 1989), which contain convenient auxotrophic markers for genetic manipulation. The strains used are listed in Table 1.

TABLE 1
Strains used herein: relevant genes in bold

| Name | Genotype | Reference |
|---|---|---|
| YPH500 | MATα ura3-52 lys2-801_amber ade2-101_ochre trp1- | Sikorski & |
| | Δ63 his3-Δ200 leu2-Δ1 | Hieter, |
| | | 1989 |
| YZGA1208 | MATα ura3-52 lys2-801_amber ade2-101_ochre trp1- | Present |
| | Δ63 his3-Δ200 leu2-Δ1snq2::hisG pdr12::hisG | study |
| | yor1::hisG-URA3-hisG | |
| YRU74 | MATα ura3-52 lys2-801_amber ade2-101_ochre trp1- | Present |
| | Δ63 his3-Δ200 leu2-Δ1 | study |
| | snq2::hisG pdr12::hisG yor1::hisG | |
| YRU94* | MATα ura3-52 lys2-801_amber ade2-101_ochre trp1- | Present |
| | Δ63 his3-Δ200 leu2-Δ1 | study |
| | snq2::hisG pdr12::hisG yor1::hisG | |
| | cka2Δ::loxP-URA3-loxP lcb3Δ::loxP-LEU2-loxP | |
| YRU94ML | MATα ura3-52 lys2-801_amber ade2-101_ochre trp1- | Present |
| | Δ63 his3-Δ200 leu2-Δ1 | study |
| | snq2::hisG pdr12::hisG yor1::hisG | |
| | cka2Δ::loxP Δlcb3Δ::loxP | |
| YTKT1 | MATα ura3-52 lys2-801_amber ade2-101_ochre trp1- | Present |
| | Δ63 his3-Δ200 leu2-Δ1 | study |
| | snq2::hisG pdr12::hisG yor1::hisG cka2Δ::loxP | |
| | lcb3Δ::loxP vps51Δ::loxP-KLLEU-loxP | |
| YTKT33 | MATα ura3-52 lys2-801_amber ade2-101_ochre trp1- | Present |
| | Δ63 his3-Δ200 leu2-Δ1 | study |
| | snq2::hisG pdr12::hisG yor1::hisG | |
| | cka2Δ::loxP lcb3Δ::loxP vps51Δ::loxP | |

Gene disruptions due to marker insertion and optional small deletion are indicated by ::, full gene deletions are indicated by the A in connection with the gene designation.

Mutant strains with disrupted PDR genes were provided by Prof. Karl Kuchler (Medical University Vienna). The plasmid, pDK30 (Sikorski R S and Hieter P, 1989) for disruption of the plasma membrane localized MRP protein encoded by YOR1 (yeast oligomycin resistance) was provided by Prof. Scott Moye-Rowley (University of Iowa).

Weak acid transporter PDR12 (Piper P et al, 1998) and the strongly expressed PDR class ABC transporter SNQ2 which is conferring resistance to multiple anionic substrates including oxalic acid (Cheng V et al, 2007) might have overlapping specificity in active efflux of FB₁. It was started with an snq2 pdr12 strain, YRE108 (Emerson L R et al., 2004) provided by Prof. K. Kuchler. After removal of the pdr12::hisG-URA3-hisG marker with 5-FOA, YOR1 was inactivated. A triple mutant (yor1 snq2 pdr12) named YRU74 was generated, by disrupting the YOR1 gene in YRE108 with the yor1::hisG-URA3-hisG plasmid pDK30.

For further gene deletions and subsequent marker removal a plasmid system based on heterologous Kluyveromyces lactis URA3 and LEU2 genes flanked by loxP sites was used (Gueldener U. et al, 2002). For gene disruptions PCR fragments were generated with long oligonucleotides (see Table 2), resulting in markers flanked by homologous sequences. Screening of obtained yeast transformants was done with flanking primers in combination with internal primers located in the selection markers.

TABLE 2
Primers used herein: Long primers for deletion using the Gueldener
plasmids (Gueldener, 2002) are indicated by d(xyzN), the constant parts priming in the
vector are underlined. Flanking primers for confirmation of correct integration are
designated scr. They were combined with primers in the selection markers (e.g pUG72
FWD or REV).

| Oligo | SEQ ID NO: | Sequence |
|---|---|---|
| dcka2-FW | 9 | ATGCCATTACCTCCGTCAACATTGAACCAGAAATCTAA |
| | | TAGAGTCTCGTACGCTGCAGGTCGAC |
| dcka2-RV | 10 | TTATTCAAACTTCGTTTTGAAAAACTTATGATCCATAGC |
| | | CTCCTTCGCATAGGCCACTAGTGGATCT |
| cka2_scr-FW | 11 | CAAAGTTGGATATCCCTAATGACC |
| cka2_scr-RV | 12 | TTACTCAAAAGGTAAATGGCTCTCT |
| delo3-FW | 13 | ATGAACACTACCACATCTACTGTTATAGCAGCAGTTGC |
| | | CGACCAGTCGTACGCTGCAGGTCGAC |
| delo3-RV | 14 | TTAAGCTTTCCTGGAAGAGACCTTGGTGTTAGAGGTC |
| | | TTGACACCCGCATAGGCCACTAGTGGATCT |
| elo3_scr-FW | 15 | ATTCACCGTCAGAGGGATTTG |
| elo3_scr-RV | 16 | GTTCAAGATAATGCAATGTCAGTCA |
| dlcb3-FW | 17 | ATGGTAGATGGACTGAATACCTCGAACATTAGGAAAA |
| | | GAGCCAGGTCGTACGCTGCAGGTCGAC |
| dlcb3-RV | 18 | TTATGCTATATTTAAGAGGGAAAATAGGACGGGGCTG |
| | | CACATTACCGCATAGGCCACTAGTGGATCT |
| lcb3_scr-FW | 19 | AAGCCTACGTTTTGGACTCTCA |
| lcb3_scr-RV | 20 | CATGGCCAGCACTATTTTCA |
| pUG72-REV | 21 | GCGTTTACCGTATCGCAGAATGG |
| pUG72-FWD | 22 | ATGGCCCAATCACAACCACATCTTAG |
| pUG73-REV | 23 | ATGCTATCGCCAAGGCTGTCAAGG |
| pUG73-FWD | 24 | GACACCTTCATCACCTAATTTCTCTTCAAC |
| VPS51_KO_pUG | 25 | GCGTATTTGCGGTGAGACGGAATCTGACGAGGATATT |
| _767_fw | | AAGTACAGCAGCTGAAGCTTCGTACGC |
| VPS51_KO_pUG | 26 | TCTCGAAGGAAGTGTTCGGTGAAAGCCACGATATGCC |
| _767_rv | | GCTGGAAAGCATAGGCCACTAGTGGATCTG |
| VPS51_KO_ | 27 | ATAGGTGAGGCTCTGCATAG |
| upstream_ | | |
| outside | | |
| VPS51_KO_ | 28 | CGAAGGAAGTGTTCGGTGAAAGC |
| downstream_ | | |
| outside | | |
| KILEU2 rev | 29 | GTTAGAAATGTCTTGGATGCAGGTG |
| KILEU2fwd | 30 | CAGCAATGGCATTCAAGACCTTA |
| SmFumD_fw_ | 31 | tcccaacgaccgaaaacctgtattttcagggatccATGAAAGAACACC |
| Leader | | AGTGTAG |
| SmFumD_fw_no | 32 | tcccaacgaccgaaaacctgtattttcagggatccATGGCTCAAACTG |
| Leader_MAQ | | ACGACCCAAA |
| SmFumD_rv | 33 | gtcttcaggagcgagttctggctggcttgcacgtgTTACTTGGATGGTT |
| | | GACAAG |
| yor1ver_fw | 34 | GGTTACGCTATTGGTGCATG |
| yor1ver_rv | 35 | GCAAGGAAAGTGACCAATG |

The constant part annealing to the Guelder plasmids pUG72 and pUG73 is underlined. Small letter bases in FumD primers are for Gibson assembly.

First single deletions of the candidate genes CKA2, and LCB3 were generated, using the loxP-KIURA3-loxP cassette (pUG72). LCB3 encodes a long-chain base-1-phosphate phosphatase involved in incorporation of exogenous long chain bases into sphingolipids (https://www.yeastgenome.org/locus/S000003670). Subsequently, double mutants were obtained. The respective PCR fragments with overhangs for the second transformation were generated by using loxP-KILEU2-loxP plasmid (pUG73). In contrast to the report that lcb3 is synthetic lethal with cka2, (Kobayashi S D. and Nagiec M M, 2003), (PCR confirmed) lcb3 cka2 double mutants in both directions (strains YRU93 and YRU94) were obtained, which showed only slightly increased FB₁ sensitivity.

The strain YRU94 was selected for further work. It was initially slow-growing and showed frequent loss of respiratory growth (and loss of the red ade2 pigmentation after recovery from storage at −80° C.). A stable clone from a large colony was selected on YPG (with glycerol as carbon source). This strain, named YRU94* was used for further work. In this strain the markers were removed by a galactose inducible PGAL1-Cre recombinase on a plasmid (pOS4a) with ADE2 as selection marker. The plasmid was subsequently lost.

In the “marker-less (ML)” strain YRU94ML (see Table 1) the gene VPS51 was subsequently inactivated with a vps51::loxP-KILEU2-loxP construct, and further enhanced sensitivity to FB₁ was observed on YPD medium. The vps51 mutants are defective in recycling of sphingolipids (transport back to Golgi), which are degraded in the vacuole instead (Olson D K et al., 2015). Upon growth on YPGal (galactose) the pOS4a plasmid was lost, but the LEU2 disruption marker retained in the analyzed candidates. One of them, strain YTKT1, was used as an intermediate to test certain candidate detoxification genes. To free up also the leu2 marker for use in transformation YTKT1 was transformed with the URA3-PGAL1-Cre plasmid, pBS49 (Sauer B, 1987), and after growth on YPGal both marker removal and plasmid loss were achieved. The resulting bioassay strain YTKT33 has the genotype:
Matα ura3-52 lys2-801_amber ade2-101_ochre trp1-Δ63 his3-Δ200 leu2-Δ1
snq2::hisG pdr12::hisG yor1::hisG cka2Δ: loxP lcb3Δ: loxP vps51Δ: loxP

### Example 2: Increased Sensitivity of Engineered Strains (YPD Medium)

Comparison of Growth Curves with Increasing FB₁ Concentrations in YPD Medium.

The sensitive yeast strains YRU74, YRU94ML and YTKT33 were grown in YPD (1% yeast extract, 2% peptone, 2% glucose) alongside the original resistant YPH500 strain. They were exposed to different concentrations of a crude fumonisin stock (FB₁+FB₂+FB₃, measured via LC-MS/MS) containing >70% FB₁. The concentrations used during the experiment were calculated based on the measured FB₁. Strain inoculum was pipetted into a microtiter well plate, which was put into an incubator for 24 h where the optical density (OD600) was measured to monitor growth. Yeast strains were diluted to an OD600 of 0.1 after reaching exponential growth and had an initial OD600 of ˜0.05 when diluted 1:1 with the FB₁-YPD mixture inside the well plate. 11 different concentrations of FB₁ were used during the experiment. The concentrations were prepared by making a 0.6N dilution series via pipetting before adding the inoculum (see microtiter well plate experimental set-up). Replicates were used for each strain and the growth after 24 h analysed by calculating the average of these replicates alongside standard deviation using excel. On the x-axis, the graph shows the final fumonisin concentration in μM that the strains were exposed to, while the y-axis shows the inhibition of growth in %. YTKT33 of well 12 (containing only YPD) was set at 100% and used to calculate the growth relative to YTKT33 of both the other strains and YTKT33 exposed to different FB₁ concentrations.

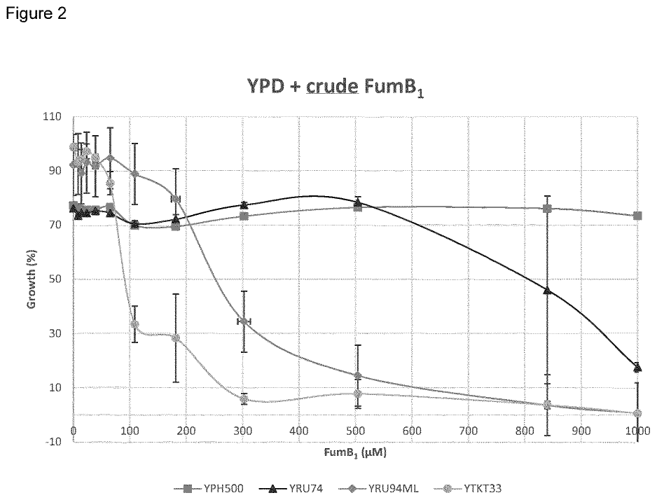

FIG. 2 shows the FB₁ sensitivity of YPH500 (control strain) and YRU74 (snq2 pdr12 yor1) and YRU94ML (snq2 pdr12 yor1 cka2Δ, lcb3Δ) and YTKT33 (snq2 pdr12 yor1 cka2Δ lcb3Δ vps51Δ) in YPD medium with increasing concentrations of crude FB₁.

In a further setting, the sensitive yeast strains YRU74, YRU94ML and YTKT33 were grown in YPD (1% yeast extract, 2% peptone, 2% glucose) overnight alongside the original resistant YPH500 strain. The next day, they were rediluted to an OD600 of 0.1. After reaching exponential growth (>0.3) they were diluted to an OD of 0.1 and diluted further (1:10 and 1:100) for spottings on YPD agar plates alongside containing different concentrations of the crude fumonisin stock (FB₁+FB₂+FB₃, measured via LC-MS/MS) containing >70% FB₁. The concentrations used for the prepared FB₁ plates were calculated based on the measured FB₁. 3 μl of each strain/dilution was pipetted onto the plates and left to grow at 30° C. for 5 days. The photos of these plates are shown above. Each line shows a different strain grown at different concentrations of FB₁. Three different dilutions of each strain were spotted on every plate. The first spot shows the strain diluted to an OD of 0.1 while the second spot shows the 1:10 dilution (OD=0.01) and the third one the 1:100 one (OD=0.001). Both YRU94ML and YTKT33 growth is inhibited at a concentration of 250 μM FB₁.

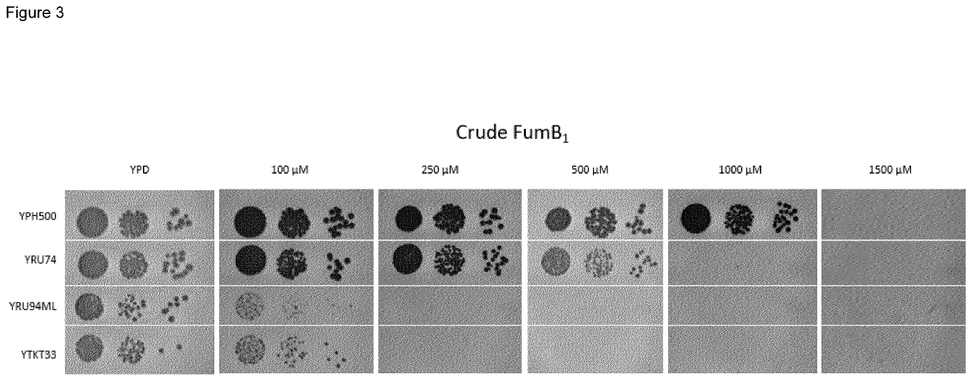

FIG. 3 shows FB₁ sensitivity of YPH500, YRU74, YRU94ML and YTKT33 on YPD agar plates with increasing concentrations of crude FB₁.

### Example 3: Increased Sensitivity of Engineered Strains (SC Medium)

Comparison of Growth Curves with Increasing FB₁ Concentrations in SC Medium.

The sensitive yeast strains YRU74, YRU94ML, YTKT33 and the original yeast strain YPH500 were grown in in Synthetic Complete (SC) media (0.67% Bacto-yeast nitrogen base w/o amino acids, 2% glucose, all necessary supplements added) overnight. They were exposed to different concentrations of a crude Fumonisin stock (FB₁+FB₂+FB₃, measured via LC-MS/MS) containing >70% FB₁. The concentrations used during the experiment were calculated based on the measured FB₁. Strain inoculum was pipetted into a microtiter well plate, which was put into an incubator for 24 h where the optical density (OD600) was measured to monitor growth. Yeast strains were diluted to an OD600 of 0.1 after reaching exponential growth and had an initial OD600 of ˜0.05 when diluted 1:1 with the FB₁-YPD mixture inside the well plate. 11 different concentrations of FB₁ were used during the experiment. The concentrations were prepared by making a 0.6^{N} dilution series via pipetting before adding the inoculum (see microtiter well plate experimental set-up). Replicates were used for each strain and the growth after 24 h analysed by calculating the average of these replicates alongside standard deviation using excel. On the x-axis, the graph shows the final Fumonisin concentration in UM that the strains were exposed to, while the y-axis shows the inhibition of growth in %. YTKT33 of well 12 (containing only YPD) was set at 100% and used to calculate the relative growth of both the other strains and YTKT33 exposed to different FB₁ concentrations.

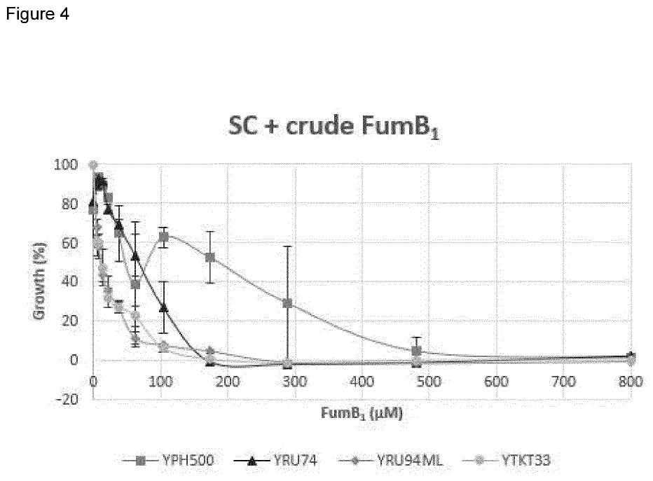

FIG. 4 shows the FB₁ sensitivity of YPH500, YRU74, YRU94ML and YTKT33 in SC medium with increasing concentrations of crude FB₁.

In a further setting, sensitive yeast strains YRU74, YRU94ML, YTKT33 and the original yeast strain YPH500 were transformed with the empty pYes2-P_{TEF1} vector containing the marker URA3, so that they could grow on Synthetic Complete media lacking uracil (SC-URA). They were grown in SC-URA overnight and rediluted to an OD600 of 0.1 in the morning. After reaching exponential growth (OD600>0.3), they were diluted again for spottings on plates containing 87,88% pure FB₁. 3 μl of each strain/dilution was pipetted onto the plates and left to grow at 30° C. for 5 days. The photos of these plates are shown above. Each line shows a different strain grown at different concentrations of FB₁. Three spots were spotted on each plate for every strain. The first spot shows the strain diluted to an OD600 of 0.1 while the second spot shows the 1:10 dilution (OD600=0.01) and the third one the 1:100 one ((OD600=0.001). YTKT33 growth is inhibited at ˜100 μM FB₁.

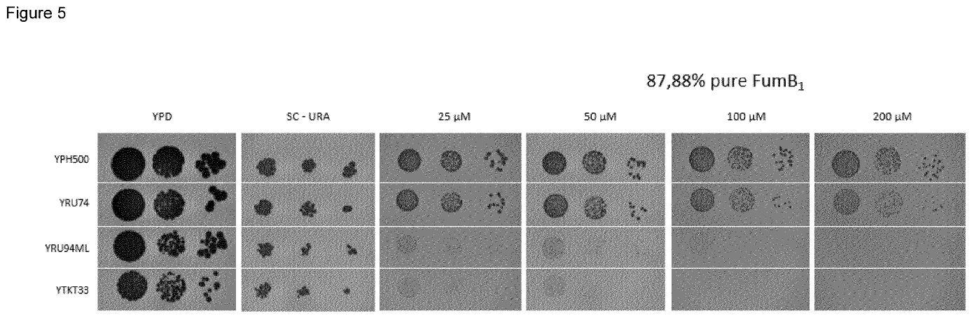

FIG. 5 shows the FB₁ sensitivity of YPH500 and YRU74 (control strains) and YRU94ML (Δsnq2, Δpdr12, Δyor1, Δcka2, Δlcb3) and YTKT33 (Δsnq2, Δpdr12, Δyor1, Δcka2, Δlcb3, Δvps51) on SC-URA agar plates (lacking uracil) with increasing concentrations of crude FB₁.

### Example 4: Detecting of Fumonisin Detoxification Genes

The Sphingopyxis sp. MTA144 fumD esterase gene (=candidate), the ancestor (Heinl S et al. 2010) of the optimized fumonisin biotransforming enzyme, “FumZyme®”, from Biomin, was provided by Dr. Dieter Moll (Biomin GmbH) and expressed without leader sequence but with an N-terminal 6×HIS tag in the sensitive strain YTKT1 (relevant genotype snq2::hisG pdr12::hisG yor1::hisG cka2Δ::loxP lcb3Δ::loxP vps51Δ::loxP-KLLEU-loxP).

Likewise, the fumonisin amine oxidase (AspAmOx) form Aspergillus (Garnham C et al., 2020) was obtained as synthetic codon-optimized gene and expressed in the sensitive yeast strain YTKT1 of the invention, with an N-terminal 6×HIS-tag, and also conferred resistance.

The plasmids described by Janevska et al., 2020, were kindly provided by Dr. Vito Valiante. Plasmid pYes2-PTEF1 (=empty vector, TEF1 promoter) was used to express different genes assumed to confer resistance against FB₁. Candidate genes were coned behind the TEF1 promoter into that plasmid and subsequently transformed into the sensitive yeast strain YTKT33 for testing.

SC-URA plates were supplemented with different FB₁ concentrations (as seen above) and spotted with 3 μl of the dilutions (OD600=0.1, 0.01 and 0.001) used for each culture. YOR1 (with endogenous promoter on an episomal (multicopy 2 μm) plasmid) and FvCER1 (positive control, resistance conferring ceramide synthase from Fusarium verticillioides) were used as controls. Sphingopyxis sp. MTA144 fumD esterase gene (“FumZyme” ancestor from Biomin) showed resistance both with and without the leader sequence (noL). Fumonisin amine oxidase from Aspergillus (AspAmOx) also confers weak resistance similar to YOR1 and the FumD esterase gene with the leader sequence.

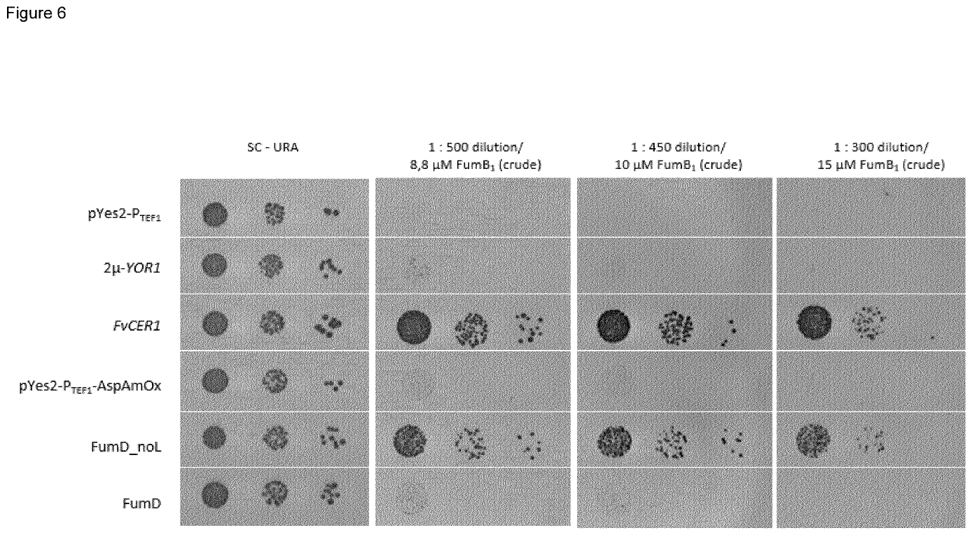

FIG. 6 shows the FB₁ sensitivities of the candidate genes YOR1, FvCER1, AspAmOx (Fumonisin amine oxidase from Aspergillus), and FumD esterase.

### Example 5: Generation of Inhibition Zone by Fumonisin-Sensitive Strain YTKT33

Strain YTKT33 was mixed into SC-agarose medium (42° C.), and paper disks were placed on the solidified medium.

Incubation of YTKT33 in the presence of water (control) or using the respective wild-type strain (YPH500) in the presence of fumonisin did not result in the development of an inhibition zone.

In contrast, incubation of YTKT33 in the presence of crude extract of F. verticillium from autoclaved maize gave an inhibition zone after 3 days incubation.

When in agar containing YTKT33 the fumonisin degrading Fumzyme® was placed close to the fumonisin disk, the detoxification enzyme was diffusing into the inhibition zone, and due to degradation of the toxin a distorted (one sided) inhibition zone was formed).

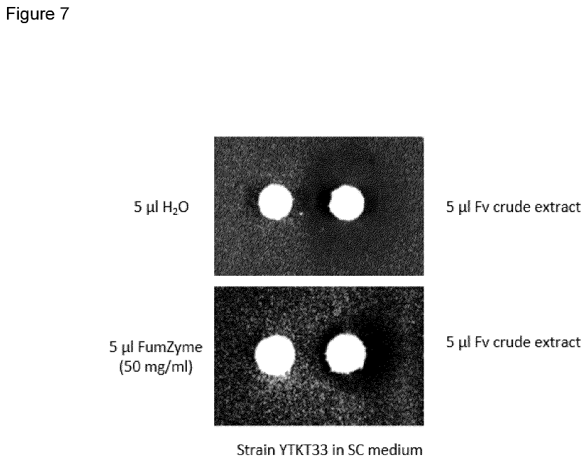

FIG. 7: Strain YTKT33 was mixed into SC-agarose medium (42° C.), and paper disks were placed on the solidified medium. Upper raw: Left-water control. Right-Crude extract of F. verticillium from autoclaved maize was added (giving an inhibition zone) after 3 days incubation. Lower row: Left-Fumonisin degrading Fumzyme was added, diffusing into the inhibition zone from fumonisin added on the right (leading to the distorted shape of the halo).

### Example 6: Testing Sensitivity of YTKT33 to Different B-Type Fumonisins

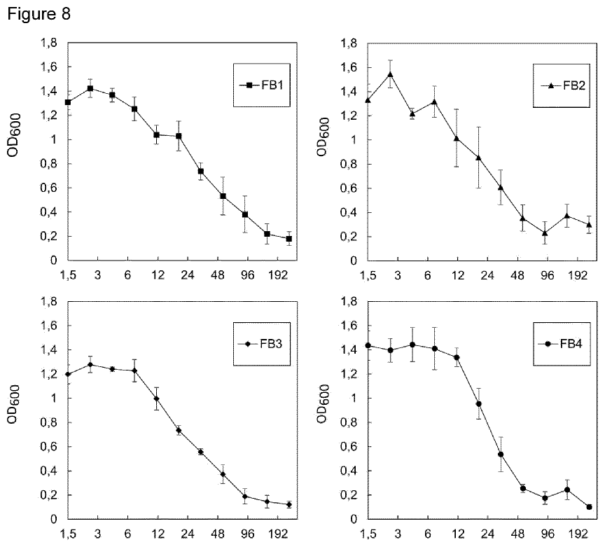

The sensitive yeast strain YTKT33 was grown in SC medium and exposed to different concentrations of FB₁, FB₂, FB₃ and FB₄. Strain inoculum was pipetted into a microtiter well plate with 0.6^{N} dilutions of the respective fumonisins. After 24 h at 30° C. the optical density at 600 nm (OD600) was measured to monitor growth. The blank (medium without yeast) was subtracted from the measured OD600 values. The results are shown in FIG. 8. On the x-axis, the graphs show the concentration of the respective fumonisins in UM (log 2 scaled). Means and standard deviations were calculated from 4 replicates.

### REFERENCES

Abbas H K et al., Biological activities of fumonisins, mycotoxins from Fusarium moniliforme, in jimsonweed (Datura stramonium L.) and mammalian cell cultures, TOXICON, 1993, vol. 31(3), pages 345-353
Abolmaali S. et al., Engineered bakers yeast as a sensitive bioassay indicator organism for the trichothecene toxin deoxynivalenol, JOURNAL OF MICROBIOLOGICAL METHOOS, 2008, vol. 72(3), p. 306-312
Bauer B E et al, Inventory and function of yeast ABC proteins: about sex, stress, pleiotropic drug and heavy metal resistance Biochim Biophys Acta. 1999 Dec. 6; 1461(2): 217-36. doi: 10.1016/s0005-2736(99) 00160-1.
Burgess K. M. N. et al., Mechanistic Insight into the Biosynthesis and Detoxification of Fumonisin Mycotoxins, ACS CHEMICAL BIOLOGY, 2016, vol. 11(9), p. 2618-2625
Cheng V et al, Genome-wide screen for oxalate-sensitive mutants of Saccharomyces cerevisiae, Appl Environ Microbiol. 2007 September; 73(18): 5919-27. doi: 10.1128/AEM.02843-06. Epub 2007 Jul. 20.
Emerson L R et al., The sensitivities of yeast strains deficient in PDR ABC transporters, to quinoline-ring antimalarial drugs, Ann Trop Med Parasitol. 2004 September; 98(6): 643-9. doi: 10.1179/000349804225021523.
Fröhlich F et al The GARP complex is required for cellular sphingolipid homeostasis eLife. 2015; 4: e08712. doi: 10.7554/eLife.08712
Garnham C P et al Identification and Characterization of an Aspergillus niger Amine Oxidase that Detoxifies Intact Fumonisins, J Agric Food Chem. 2020 Nov. 25; 68(47): 13779-13790. doi: 10.1021/acs.jafc.0c04504. Epub 2020 Nov. 11.
Gelderblom W C et al., Toxicity and carcinogenicity of the Fusarium moniliforme metabolite, fumonisin B1, in rats, Carcinogenesis, 1991 July; 12(7): 1247-51. doi: 10.1093/carcin/12.7.1247
Gelderblom W C et al, The cancer-initiating potential of the fumonisin B mycotoxins, Carcinogenesis. 1992 March; 13(3): 433-7. doi: 10.1093/carcin/13.3.433.
Gelderblom W C et al Structure-activity relationships of fumonisins in short-term carcinogenesis and cytotoxicity assays, Food Chem Toxicol 1993 June; 31(6): 407-14. doi: 10.1016/0278-6915(93) 90155-r.
Gietz R D et al Yeast studies on the transformation of intact yeast cells by the LiAc/SS-DNA/PEG procedure. 1995 Apr. 15; 11(4): 355-60. doi: 10.1002/yea.320110408.
Glenn A E et al, Transformation-mediated complementation of a FUM gene cluster deletion in Fusarium verticillioides restores both fumonisin production and pathogenicity on maize seedlings, Mol Plant Microbe Interact 2008 January; 21(1): 87-97. doi: 10.1094/MPMI-21-1-0087.
Gueldener U et al., A second set of loxP marker cassettes for Cre-mediated multiple gene knockouts in budding yeast, Nucleic Acids Res. 2002 Mar. 15; 30(6): e23. doi: 10.1093/nar/30.6.e23.
Harrison L R et al., Pulmonary edema and hydrothorax in swine produced by fumonisin B₁, a toxic metabolite of Fusarium moniliforme, J Vet Diagn Invest. 1990 July; 2(3): 217-21. doi: 10.1177/104063879000200312.
Hartl M and Humpf H U Toxicity assessment of fumonisins using the brine shrimp (Artemia salina) bioassay, Food Chem Toxicol. 2000 December; 38(12): 1097-102. doi: 10.1016/s0278-6915(00) 00112-5.
Hartinger D et al Enzyme characteristics of aminotransferase Fuml of Sphingopyxis sp. MTA144 for deamination of hydrolyzed fumonisin B₁, Appl Microbiol Biotechnol. 2011 August; 91(3): 757-68. doi: 10.1007/s00253-011-3248-9.
Heinl S et al Degradation of fumonisin B₁ by the consecutive action of two bacterial enzymes J Biotechnol. 2010 Jan. 15; 145(2): 120-9. doi: 10.1016/j.jbiotec.2009.11.004. Epub 2009 Nov. 14.
Hirabayashi Y et al Sphingolipid Biology, 2006, Springer Verlag, ISBN-104-431-34198-6
Hlywka J J et al The use of the chicken embryo screening test and brine shrimp (Artemia salina) bioassays to assess the toxicity of fumonisin B₁ mycotoxin, Food Chem Toxicol. October-November 1997; 35(10-11): 991-9. doi: 10.1016/s0278-6915(97) 87268-7
Jakociunas T et al., Multiplex metabolic pathway engineering using CRISPR/Cas9 in Saccharomyces cerevisiae, 2015, Metabolic Engineering 28(2015) 213-222
Janevska S et al Self-Protection against the Sphingolipid Biosynthesis Inhibitor Fumonisin B₁ Is Conferred by a FUM Cluster-Encoded Ceramide Synthase mBio. 2020 Jun. 16; 11(3): e00455-20. doi: 10.1128/mBio.00455-20.
Karlin S and Altschul S F, Methods for assessing the statistical significance of molecular sequence features by using general scoring schemes Proc Natl Acad Sci USA. 1990 March; 87(6): 2264-8. doi: 10.1073/pnas.87.6.2264.
Karlin S and Altschul S F, Applications and statistics for multiple high-scoring segments in molecular sequences Proc Natl Acad Sci USA. 1993 Jun. 15; 90(12): 5873-7. doi: 10.1073/pnas.90.12.5873.
Kobayashi S D. and Nagiec M M, Ceramide/long-chain base phosphate rheostat in Saccharomyces cerevisiae: regulation of ceramide synthesis by Elo3p and Cka2p, Eukaryot Cell. 2003 April; 2(2): 284-94. doi: 10.1128/EC.2.2.284-294.2003.
Lemke S L et al Deamination of Fumonisin B₁ and Biological Assessment of Reaction Product Toxicity, Chem. Res. Toxicol. 2001, 14, 1, 11-15
Louis E Historical evolution of laboratory strains of Saccharomyces cerevisiae, 2016, Cold Spring Harbor protocols, 585-593, doi: 10.1101/pdb.top077750
Luttgeharm K D et al Substrate specificity, kinetic properties and inhibition by fumonisin B₁ of ceramide synthase isoforms from Arabidopsis, Biochem J 2016 Mar. 1; 473(5): 593-603. doi: 10.1042/BJ20150824. Epub 2015 Dec. 3.
Mahé et al, The ATP binding cassette transporters Pdr5 and Snq2 of Saccharomyces cerevisiae can mediate transport of steroids in vivo. J Biol Chem. 1996 Oct. 11; 271(41): 25167-72. doi: 10.1074/jbc.271.41.25167.
Piper et al., The pdr12 ABC transporter is required for the development of weak organic acid resistance in yeast. EMBO J. 1998 Aug. 3; 17(15): 4257-65. doi: 10.1093/emboj/17.15.4257.
Marasas W F et al. Leukoencephalomalacia in a horse induced by fumonisin B₁ isolated from Fusarium moniliforme, Onderstepoort J Vet Res. 1988 December; 55(4): 197-203.
Matsuura A et al Gene Apg1p, a novel protein kinase required for the autophagic process in Saccharomyces cerevisiae. 1997 Jun. 19; 192(2): 245-50. doi: 10.1016/s0378-1119(97) 00084-x.
Mckean C et al., Comparative acute and combinative toxicity of aflatoxin B₁ and fumonisin B₁ in animals and human cells, 2006, Food Chem Toxicol. 2006 June; 44(6): 868-76. doi: 10.1016/j.fct.2005.11.011.
Merrill A. H. Jr., et al. Fumonisin toxicity and sphingolipid biosynthesis, Adv Exp Med Biol. 1996; 392:297-306. doi: 10.1007/978-1-4899-1379-1_25.
Missmer S A et al., Exposure to fumonisins and the occurrence of neural tube defects along the Texas-Mexico border, Environ Health Perspect. 2006 February; 114(2): 237-41. doi: 10.1289/ehp.8221
Miyake R. et al., Transporter-Driven Engineering of a Genetic Biosensor for the Detection and Production of Short-Branched Chain Fatty Acids in Saccharomyces cerevisiae, FRONTIERS IN BIOENGINEERING AND BIOTECHNOLOGY, 2022, vol. 10
Molina-Pintor I B et al., An update on genotoxic and epigenetic studies of fumonisin B₁ 2021, World Mycotoxin Journal: 15(1)-Pages: 57-72
Myers E W and Miller W Optimal alignments in linear space, Comput Appl Biosci. 1988 March; 4(1): 11-7. doi: 10.1093/bioinformatics/4.1.11
Olson D K et al., Taming the sphinx: Mechanisms of cellular sphingolipid homeostasis Biochim Biophys Acta. 2016 August; 1861(8 Pt B): 784-792. doi: 10.1016/j.bbalip.2015.12.021. Epub 2015 Dec. 30.
Paumi C M t al., ABC Transporters in Saccharomyces cerevisiae and Their Interactors: New Technology Advances the Biology of the ABCC (MRP) Subfamily, MICROBIOLOGY AND MOLECULAR BIOLOGY REVIEWS, December 2009, p. 577-593 Vol. 73, No. 4 doi: 10.1128/MMBR.00020-09
Piper P et al., The pdr12 ABC transporter is required for the development of weak organic acid resistance in yeast, EMBO J. 1998 Aug. 3; 17(15): 4257-65. doi: 10.1093/emboj/17.15.4257.
Poppenberger B et al., Detoxification of the Fusarium mycotoxin deoxynivalenol by a UDP-glucosyltransferase from Arabidopsis thaliana J Biol Chem. 2003 Nov. 28; 278(48): 47905-14. doi: 10.1074/jbc.M307552200. Epub 2003 Sep. 11.
Rego A et al., The yeast model system as a tool towards the understanding of apoptosis regulation by sphingolipids, FEMS Yeast Res. 2014 February; 14(1): 160-78. doi: 10.1111/1567-1364.12096.
Qie L et al., Identification of a Saccharomyces Gene, LCB3, Necessary for Incorporation of Exogenous Long Chain Bases into Sphingolipids*, 1997, JBC, 272, 26, 16110-16117
Renaud J B et al., 2021, Structure activity relationship for fumonisin phytotoxicity, Chem Res. Toxicol., 1604-1611
Rogers B at al., The pleitropic drug ABC transporters from Saccharomyces cerevisiae, JOURNAL OF MOLECULAR MICROBIOLOGY AND BIOTECHNOLOGY, 2001, vol. 3(2), p. 207-214
Sauer B, Functional expression of the cre-lox site-specific recombination system in the yeast Saccharomyces cerevisiae, Mol Cell Biol. 1987 June; 7(6): 2087-96. doi: 10.1128/mcb.7.6.2087-2096.1987.
Rothstein R, Targeting, disruption, replacement, and allele rescue: integrative DNA transformation in yeast, 1991, Methods in Enzymology 194:281-302
Sherman F., Methods in Yeast Genetics, 1981, Cold Spring Harbor Laboratory
Sikorski R S and Hieter P, A system of shuttle vectors and yeast host strains designed for efficient manipulation of DNA in Saccharomyces cerevisiae, Genetics. 1989 May; 122(1): 19-27. doi: 10.1093/genetics/122.1.19.
Stone J M et al., Simulation of Fungal-Mediated Cell Death by Fumonisin B₁ and Selection of Fumonisin B₁-Resistant (fbr) Arabidopsis Mutants 2000, Plant Cell. 2000 October; 12(10): 1811-1822
Sun Lei et al., Effects of Disruption of Five FUM Genes on Fumonisin Biosynthesis and Pathogenicity in Fusarium proliferatum, Toxins (Basel) 2019 Jun. 7; 11(6): 327. doi: 10.3390/toxins11060327.
TAKAGI K. et al., Involvement of Golgi-associated retrograde protein complex in the recycling of the putative Dnf aminophospholipid flippases in yeast, BIOCHEMICAL AND BIOPHYSICAL RESEARCH COMMUNICATIONS, 2012, vol. 417(1), p. 490-494
Voss K A et al., Carcinogenicity and mechanism of action of fumonisin B₁: a mycotoxin produced by Fusarium moniliforme (=F. verticillioides). Cancer Detect Prev. 2002; 26(1): 1-9. doi: 10.1016/s0361-090x(02) 00011-9

1. A fumonisin-sensitive yeast comprising disrupted or deleted genes SNQ2, PDR12, YOR1, CKA2, and LCB3, and optionally any one of VPS51, VPS52, or VPS53.

2. The fumonisin-sensitive yeast of claim 1, wherein said yeast comprises disrupted or deleted genes having a sequence identity of at least 50% with any one of SEQ ID NOs: 1, 2, 3, 4, and 5.

3. The fumonisin-sensitive yeast of claim 1, wherein said yeast is of the phylum Ascomycota, preferably a Saccharomyces cerevisiae strain.

4. The fumonisin-sensitive yeast of claim 1, comprising selectable marker genes, preferably resistance or auxotrophic markers, specifically selected from the group consisting of LEU2, URA3, LYS2, ADE2, TRP1, and HIS3.

5. The fumonisin-sensitive yeast of claim 1, wherein genes SNQ2, PDR12, YOR1, CKA2, LCB3, and VPS51 are disrupted or deleted.

6. The fumonisin-sensitive yeast of claim 1, wherein said yeast strain is a Saccharomyces cerevisiae mutant strain comprising the genotype snq2::hisG pdr12::hisG yor1::hisG cka2Δ::loxP lcb3Δ::loxP vps51Δ::loxP.

7. The fumonisin-sensitive yeast of claim 1, wherein the fumonisin is Fumonisin B₁ (FB₁), FB₂, FB₃, FB₄, FB₅, FB₆, FC₁, FC₂, FC₃, FC₄, or isomers thereof, or any combination thereof.

8. Use of the fumonisin-sensitive yeast of claim 1 for screening fumonisin detoxifying compounds or enzymes, or for toxicological studies.

9. A method for detecting inhibitory activity of fumonisins in a sample, comprising the steps of
i. contacting the fumonisin-sensitive yeast of claim 1 with the sample;
ii. determining the growth of said yeast in the presence and absence of said sample; wherein
iii. reduced growth indicates the presence of fumonisin in the sample.

10. The method of claim 9, wherein the sample comprises plant extracts, more preferably corn extracts, or extracts of fungal cultures.

11. A method for detecting fumonisin detoxifying compounds in a sample, comprising the steps of
i. contacting the fumonisin-sensitive yeast of claim 1 with the sample;
ii. determining growth of said yeast in the presence and absence of said sample; wherein
iii. increased growth indicates the presence of fumonisin detoxification compounds in the sample.

12. The method of claim 11, wherein the sample comprises extracts of fungi, crude protein preparations, or purified proteins.

13. The method of claim 12, wherein the fumonisin detoxifying or resistance conferring compounds are polypeptides, proteins, preferably the compounds are enzymes.

14. A method for screening the fumonisin detoxification or resistance conferring activity of a heterologous polypeptide, comprising the steps of
i. introducing a heterologous DNA sequence encoding a potential fumonisin detoxification or resistance conferring polypeptide into the fumonisin-sensitive yeast of claim 1;
ii. expressing the potential fumonisin detoxification polypeptide in said yeast;
iii. determining the growth of said yeast;
iv. whereby growth in the presence of fumonisin of said yeast compared to a reference yeast lacking said heterologous DNA sequence indicates detoxification or resistance conferring activity of the polypeptide.

15. The method of claim 14, wherein the fumonisin detoxification or resistance conferring polypeptide is an enzyme, an oxidase, preferably an amine oxidase, an N-acetyltransferase, or an esterase.

16. The method of claim 14, wherein the detoxification or resistance conferring activity of one or more polypeptides is screened, wherein the one or more heterologous DNA sequence encoding said one or more polypeptides are introduced into the same fumonisin-sensitive cell or in separate fumonisin-sensitive yeasts.
